(19) United States
(12) Patent Application Publication
(10) Pub. No.: US 2021/0393664 A1
(43) Pub. Date: Dec. 23, 2021

(54) THERAPEUTIC METHODS AND COMPOSITIONS FOR TREATING ACUTE MYELOID LEUKEMIA USING DEVIMISTAT

(71) Applicant: Rafael Pharmaceuticals, Inc., Cranbury, NJ (US)

(72) Inventors: Sanjeev Luther, Cranbury, NJ (US); Timothy S. Pardee, Winston-Salem, NC (US); Jorge Jorge Cortes, Houston, TX (US)

(21) Appl. No.: 17/297,265

(22) PCT Filed: Nov. 27, 2019
(86) PCT No.: PCT/US2019/063553

§ 371 Date: May 26, 2021

Related U.S. Application Data
(60) Provisional application No. 62/773,483, filed on Nov. 30, 2018.

(51) Int. Cl. A61K 31/7068; A61K 31/136; A61K 31/20; A61K 9/00; A61P 35/02

(52) U.S. Cl. CPC A61K 31/7068; A61K 31/136; A61P 35/02; A61K 9/0019; A61K 31/20

(57) ABSTRACT

The invention provides methods, compositions, and medical kits for treating acute myeloid leukemia using devimistat in combination with cytarabine and mitoxantrone.

### CROSS-REFERENCE TO RELATED APPLICATIONS

This application is the national stage application of International (PCT) Patent Application Serial No. PCT/US2019/063553, filed Nov. 27, 2019, which claims the benefit of and priority to U.S. Provisional Patent Application Ser. No. 62/773,483, filed Nov. 30, 2018, the contents of these applications are hereby incorporated by reference.

### FIELD OF THE INVENTION

The invention provides methods, compositions, and medical kits for treating acute myeloid leukemia using (i) devimistat in combination with (ii) cytarabine and (iii) mitoxantrone.

### BACKGROUND

AML is a hematologic malignancy characterized by the accumulation of clonal myeloid progenitor cells (“blasts”) in the blood or bone marrow (Ferrara F, Schiffer C A., “Acute myeloid leukaemia in adults,” Lancet, 2013, 381, 484-495; Gutierrez S E, Romero-Oliva F A, “Epigenetic changes: a common theme in acute myelogenous leukemogenesis,” Journal of Hematology & Oncology, 2013, 6, 57; Kubal T, Lancet J E, “The thorny issue of relapsed acute myeloid leukemia,” Current Opinion in Hematology, 2013, 20, 100-106). Malignant hematopoietic stem and myeloid progenitor cells proliferate uncontrollably and disrupt normal hematopoiesis, resulting in impairments in the ability of these myeloid progenitor cells to differentiate (Konoplev S, Bueso-Ramos C E, “Advances in the pathologic diagnosis and biology of acute myeloid leukemia,” Annals of Diagnostic Pathology, 2006, 10, 39-65). An orphan disease, it has a current prevalence of approximately 40,000 individuals in the United States.

AML has a variety of cytogenetic causes and usually requires a mutation in a Class I gene that stimulates a signal transduction pathway (such as FLT3 or RAS) and in a Class II gene, typically a transcription factor such as CCAAT enhancer binding protein A (CEBPα) that prevents normal hematopoietic differentiation. Another class of commonly mutated genes in AML, referred to as Class III genes, includes epigenetic modifiers that also contribute to pathogenesis. The genetic makeup of AML can dictate the severity of the disease course (Ferrara F, Schiffer C A.; Gutierrez S E, Romero-Oliva F A, supra). To date, there are multiple cytogenetic characteristics and molecular abnormalities that have been associated with disease risk and outcomes, with risk status classified as favorable, intermediate, or poor based on the patient's cytogenetics and molecular abnormalities that have been associated with disease risk and outcomes. The risk stratification based on these features can be found in the most recent National Comprehensive Cancer Network (NCCN) guidelines (v.3 in 2019) is summarized in Table 1.

TABLE 1
Risk Stratification.
EUROPEAN LEUKEMIANET RISK
STRATIFICATION BY GENETICS IN NON-APL AML^{1, 2}

| Risk Category* | Genetic Abnormality |
|---|---|
| Favorable | t(8; 21)(q22; q22.1); RUNX1-RUNX1T1 |
| | inv(16)(p13.1q22) or t(16; 16)(p13.1; q22); CBFB-MYH11 |
| | Mutated NPM1 without FLT3-ITD or with FLT3-ITD**† |
| | Baillelic mutated CEBPA |
| Intermediate | Mutated NPM1 and FLT3-ITD [symbol] † |
| | Wild-type NPM1 without FLT-ITD of with FLT3-ITD [symbol] † |
| | (without adverse-risk genetic lesions) |
| | t(9; 11)(p21.3; q23.3); MLLT3-KMT2A‡ |
| | Cytogenetic abnormalities not classified as favorable |
| | or adverse |
| Poor/Adverse | t(6; 9)(p23; q34.1); DEK-NUP214 |
| | t(v; 11q23.3); KMT2A rearranged |
| | t(9; 22)(q34.1; q11.2); BCR-ABL1 |
| | inv(3)/(q21.3q26.2) or t(3; 3 [symbol] q21.3; |
| | q26.2); GATA2.MECOM(EVI1) |
| | −5 or del(5q); −7; −17/abn(17p) |
| | Complex karyotype, § monosonomal karyotype∥ |
| | Wild-type NPM1 and FLT3-ITD [symbol] † |
| | Mutated RUNX1¶ |
| | Mutated ASXL1¶ |
| | Mutated TP53# |

¹ Dohner H, Estey E, Grimwade D, et al. Diagnosis and management of AML in adults: 2017 ELN recommendations from an international expert panel. Blood 2017; 129: 424-447.
²Frequencies, response rates, and outcome measures should be reported by risk category, and, if sufficient numbers are available, by specific genetic lesions indicated.
*Prognostic impact of a marker is treatment-dependent and may change with new therapies.
† Low, low allelic ratio (<0.5); high, high allelic ratio (≥0.5); semi quantitative assessment of FLT3-ITD allelic ratio (using DNA fragment analysis) is determined as ratio of the area under the curve “FLT3-ITD” divided by area under the curve “FLT3-wild type”; regardless of FLT3 allelic fractions, patients should be considered for bone marrow transplant, though recent studies indicate that AML with NPM1 mutation and FLT3-ITD low allelic ratio may also have a more favorable prognosis and patients should not routinely be assigned to allogeneic HCT. FLT3 allelic ratio is not yet pervasively used, and IF not available, the presence of an FLT3 mutation should be considered high-risk unless it occurs concurrently with an NPM1 mutation, in which case it is intermediate risk. As data emerge, this measure will evolve.
‡ The presence of t(9; 11)(p21.3; q23.3) takes precedence over rare, concurrent adverse-risk gene mutations.
§ Three or more unrelated chromosome abnormalities in the absence of 1 of the WHO-designated recurring translocations or inversions, that is t(8; 21), inv(16) or t(16; 16), t(9; 11), t(v; 11) (v; q23.3), t(6; 9), inv(3) or t(3; 3); AML with BCR-ABL1.
∥ Defined by the presence of 1 single monosomy (excluding loss of X or Y) in association with at least 1 additional monosomy or structural chromosome abnormality (excluding core-binding factor AML).
¶ These markers should not be used as an adverse prognostic marker if they co-occur with favorable-risk AML subtypes.
#TP53 mutations are significantly associated with AML with complex and monosomal karyotype. [symbol]
indicates data missing or illegible when filed

AML symptoms are usually a direct result of the leukemic infiltration of the bone marrow (Lowenberg B. et al., “Acute Myeloid Leukemia,” New England Journal of Medicine, 1999, 341, 1051-1062). The hallmark sign of AML, disordered hematopoiesis, results in symptoms such as bleeding (nose bleeds, retinal hemorrhages, and/or gingival bleeding), weight loss, organomegaly, lethargy, fatigue, paleness, frequent infections, sternal tenderness, and bruising easily. Symptoms can also arise as a result of an extremely high white blood cell counts, including difficulty in breathing, confusion, or other symptoms of organ failure. Diagnosis of AML is based on a myeloblast count of at least 20% of nucleated cells in the blood or bone marrow. These leukemic myeloblasts cannot differentiate further and lead to the characteristic myeloid blast accumulation of AML (Hasserjian R P, “Acute myeloid leukemia: advances in diagnosis and classification,” International Journal of Laboratory Hematology, 2013, 35, 358-366). Because of the accumulation of malignant blasts, there is a concomitant reduction in the development and production of other normal blood cells. These deficiencies in the hematopoietic system, in conjunction with increased production of malignant cells, lead to anemia, neutropenia, thrombocytopenia, and mortality (Ferrara F, Schiffer C A., supra).

Prognosis for patients with AML is variable. Even with intensive therapy, 3-year overall survival (OS) in adults after first-line AML therapy is only 29%, 17% and 9% in patients with good, intermediate, and poor risk, respectively (Sakamoto K M, et al., “Targeting novel signaling pathways for resistant acute myeloid leukemia,” Molecular Genetics and Metabolism, 2014). Mortality rates range from 20% to 75% in patients under 60 years of age and >90% in elderly patients (Gutierrez S E, Romero-Oliva F A, supra). Recent 5- and 10-year survival estimates for AML patients are 21.4% and 18.7% respectively for all ages, with more favorable survival rates for patients under the age of 44 (Pulte D, Gondos A, Brenner H, “Expected long-term survival of patients diagnosed with acute myeloblastic leukemia during 2006-2010,” Annals of Oncology: Official Journal of the European Society for Medical Oncology/ESMO, 2010, 21, 335-341).

Treatment for AML typically consists of two stages: an induction stage and a post-remission, consolidation stage. Patients who do not receive post-remission therapy usually relapse within 4-6 months. The goals of induction therapy are reaching complete remission (CR) and restoring blood counts back to healthy levels without the need for transfusions (Milner H et al., “Diagnosis and management of acute myeloid leukemia in adults: 2017 ELN recommendations from an international expert panel, Blood, 2017, 129, 424-447). Complete remission in AML is described as the presence of <5% bone marrow blasts, absence of extramedullary disease, an absolute neutrophil count >1,000/μL, a platelet count >100,000/μL, and independence of red cell transfusions. A CR with incomplete recovery (CRi) is considered when complete remission criteria are met except for residual neutropenia (<1,000/μL) or thrombocytopenia (<100,000/μL) (Darner H et al., supra). Induction therapy strategies are influenced by individual patient characteristics such as age, performance and functional status, comorbid conditions, and known cytogenetic profile.

As per the National Comprehensive Cancer Network (NCCN), induction treatment is comprised of a seven-day infusion of cytarabine (100-200 mg/m² for 7 days) with a short infusion on days 1 through 3 of an anthracycline or anthracenedione, and is different in elderly patients (60+ years of age), as described below (www.NCCN.org., supra):
Patients <60 years old. 1) Clinical trial (preferred), or; 2) standard dose cytarabine for seven days with idarubicin or daunorubicin for three days, or; 3) standard dose cytarabine for seven days with daunorubicin and cladribine for three days, or; 4) high dose cytarabine (HiDAC) for four or six days with idarubicin or daunorubicin for three days, or; 5) fludarabine and cytarabine for four days, idarubicin for two days, and granulocyte colony stimulating factor for seven days.
Patients ≥60 years old. 1) Clinical trial, or; 2) standard dose cytarabine for seven days with idarubicin, daunorubicin, or mitoxantrone for three days, or; 3) azacytidine/decitabine, low dose cytarabine, or; 4) clofarabine and cytarabine, or; 5) best supportive care, which includes hydroxyurea and transfusion support.

Younger patients who achieve complete remission most often do so with one course, though a second course will induce complete remission in 70-75%. In patients over 60 years of age, approximately 50% will enter remission following induction therapy. Within this 50% of patients in remission, approximately 85% will subsequently relapse within 2 years resulting in a very poor prognosis (Burnett A K, “Treatment of acute myeloid leukemia: are we making progress?” Hematology Am Soc Hematol Educ Program, 2012, 2012, 1-6). However, to date, the only predictive factor for remission duration is reaching complete remission within 30 days of induction therapy (Pullarkat V, Aldoss I., “Prognostic and therapeutic implications of early treatment response assessment in acute myeloid leukemia.” Critical Reviews in Oncology/Hematology, 2015).

Once a patient achieves CR, additional therapy is needed to eradicate residual leukemia cells. This post-remission consolidation therapy may include additional cycles of intensive chemotherapy and/or autologous or allogenic bone marrow transplants. In younger patients, strategies for consolidation are based on the potential risk of relapse, with cytogenetic and molecular abnormalities representing the most significant prognostic indicators. In patients <60 years old and with good or intermediate risk cytogenetics, multiple cycles of HiDAC therapy (3-4) are the standard consolidation regimen. Those with intermediate cytogenetic risk can achieve a lower risk of relapse following hematopoietic cell transplant.

In patients >60 years old, post-remission therapy varies depending on the response achieved. Patients who achieved a CR or CRi with standard induction chemotherapy may receive further consolidation with the same agents. The use of myeloablative allogeneic hematopoietic cell transplant is limited in older patients due to significant comorbidities. For those with induction failure, a clinical trial, allogeneic hematopoietic cell transplant in the context of a clinical trial, or best supportive care are the NCCN-recommended treatment options (www.NCCN.org., supra).

Unfortunately, many patients who achieve a remission and complete consolidation therapy still have a guarded prognosis. This is driven by the fact that over 50% of patients will experience a relapse, and most of them will die from AML within a year (Bennett J M et al., “Long-term survival in acute myeloid leukemia: the Eastern Cooperative Oncology Group experience.” Cancer, 1997, 80, 2205-2209). Additionally, patients refractory to standard induction therapy have low salvage rates and high mortality. There is no consensus standard treatment for relapsed or refractory disease but most fit patients are treated with a HiDAC-based regimen (Bennett J M et al., supra). The goal of salvage therapy is to attain a second complete remission and whenever possible proceed to an allogeneic stem cell transplant. Stem cell transplant is the only curative therapy in this setting and outcomes are best when patients are in remission at the time of transplant (Bennett J M et al., supra). In patients over the age of 60 transplants are done with reduced intensity conditioning regimens making disease control at the time of transplant critical for success. As a result, achieving a second complete remission is critical for the long-term survival of these patients and any increase in the poor response rates seen in these patients will likely result in improved long-term survival.

Present standard of care regimens for AML are insufficient with less than 25% of patients surviving 5 or more years. This number is less than 10% for patients 50 years of age or older. Even with intensive therapy, 3-year OS in adults after first-line AML therapy is only 29%, 17% and 9% in patients with good, intermediate, and poor risk, respectively. Available treatment options for AML patients are venetoclax, liposomal cytarabine and daunorubicin, azacitidine, midostaurin, cyclophosphamide, cytarabine, daunorubicin, idarubicin, mitoxantrone, thioguanine, vincristine and gemtuzumab ozogamicin. Treatment with standard chemotherapies have wide range of serious toxicities. Thus, there is a clear unmet medical need for better treatment options for these acute myeloid leukemia patients. The present invention addresses this need and provides other related advantages.

### SUMMARY

The invention provides methods, compositions, and medical kits for treating acute myeloid leukemia using devimistat in combination with cytarabine and mitoxantrone. The acute myeloid leukemia may be, for example, relapsed or refractory. The patient treated according to the method of the present invention may be greater than or equal to 50 years old. The patient treated according to the method of the present invention may be greater than or equal to 60 years old. The devimistat may be formulated as a pharmaceutical composition, such as a pharmaceutical composition containing an ion pairing agent. The devimistat may be formulated as a pharmaceutical composition for administration to the patient separate from pharmaceutical compositions containing the cytarabine and mitoxantrone.

Accordingly, one aspect of the invention provides a method for treating acute myeloid leukemia. The method comprises administering to a patient in need thereof devimistat, cytarabine, and mitoxantrone pursuant to an induction cycle of 14 days, wherein during the induction cycle the devimistat is administered as a single daily dose of about 2,000 mg/m² on each of days 1, 2, 3, 4, and 5, the cytarabine is administered in five doses of about 1.0 g/m² each every 12 hours beginning on day 3, and the mitoxantrone is administered as a single daily dose of about 6 mg/m² on each of days 3, 4, and 5, in order to treat the acute myeloid leukemia. In certain embodiments the method further comprises the step of repeating the induction cycle one time.

In certain embodiments, after one induction cycle the method further comprises the step of administering to the patient devimistat, cytarabine, and mitoxantrone pursuant to an abbreviated induction cycle of 14 days, wherein during the abbreviated induction cycle the devimistat is administered as a single daily dose of about 2,000 mg/m² on each of days 1, 2, and 3, the cytarabine is administered in three doses of about 1.0 g/m² each every 12 hours beginning on day 2, and the mitoxantrone is administered as a single daily dose of about 6 mg/m² on each of days 2 and 3.

In certain embodiments, after one induction cycle optionally followed by a repeated induction cycle or an abbreviated induction cycle, the method further comprises administering to the patient devimistat, cytarabine, and mitoxantrone pursuant to a consolidation cycle of 14 days, wherein during the consolidation cycle the devimistat is administered as a single daily dose of about 2,000 mg/m² on each of days 1, 2, and 3, the cytarabine is administered in three doses of about 1.0 g/m² each every 12 hours beginning on day 2, and the mitoxantrone is administered as a single daily dose of about 6 mg/m² on each of days 2 and 3. In certain embodiments, the method further comprises the step of repeating the consolidation cycle one time.

In certain embodiments, after one induction cycle optionally followed by a repeated induction cycle or an abbreviated induction cycle, and optionally followed by one or two consolidation cycles, the method further comprises the step of administering to the patient devimistat pursuant to a maintenance cycle of 28 days, wherein during the maintenance cycle the devimistat is administered as a single daily dose of about 2,500 mg/m² on each of days 1, 2, 3, 4, and 5. In certain embodiments, the method further comprises the step of repeating the maintenance cycle at least one time.

In certain embodiments, the mitoxantrone is administered intravenously to the patient as a solution prepared from its dihydrochloride salt. In certain embodiments, the devimistat is administered intravenously to the patient as a solution prepared by the steps of (a) providing a 50 mg/mL solution of devimistat in 1 M aqueous triethanolamine; and (b) diluting the 50 mg/mL solution with sterile 5% dextrose for injection (D5W) to a concentration of about 12.5 mg/mL.

Preferably, during treatment according to the method of the present invention devimistat, cytarabine, and mitoxantrone are administered to the patient only as described herein pursuant to the induction cycle and optional induction, abbreviated induction, consolidation, and maintenance cycles and no additional devimistat, cytarabine, or mitoxantrone is administered. In addition, it is preferred that during treatment according to the method of the present invention, the patient is not administered other drugs that treatment acute myeloid leukemia.

The foregoing aspects of the invention are described in more detail, along with additional embodiments, in the detailed description below.

### DETAILED DESCRIPTION

The invention provides methods, compositions, and medical kits for treating acute myeloid leukemia with devimistat in combination with cytarabine and mitoxantrone pursuant to an induction cycle of 14 days, wherein during the induction cycle the devimistat is administered as a single daily dose of about 2,000 mg/m² on each of days 1, 2, 3, 4, and 5, the cytarabine is administered in five doses of about 1.0 g/m² each every 12 hours beginning on day 3, and the mitoxantrone is administered as a single daily dose of about 6 mg/m² on each of days 3, 4, and 5, in order to treat the acute myeloid leukemia. Various aspects of the invention are set forth below in sections; however, aspects of the invention described in one particular section are not to be limited to any particular section.

### I. Definitions

To facilitate an understanding of the present invention, a number of terms and phrases are defined below.

The terms “a,” “an” and “the” as used herein mean “one or more” and include the plural unless the context is inappropriate

The term “devimistat” refers to 6,8-bis-benzylthio-octanoic acid (CPI-613®), having the chemical structure

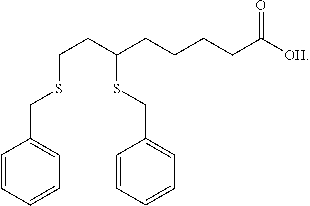 [SMILES: O=C(O)CCCCC(CCSCc1ccccc1)SCc1ccccc1]

Certain compounds contained in compositions of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention.

As used herein, the term “patient” refers to a human being in need of treatment for acute myeloid leukemia.

As used herein, the term “treating” includes any effect, e.g., lessening, reducing, modulating, ameliorating or eliminating, that results in the improvement, stabilization, or slowing progression of a condition, disease, disorder, or the like, or a symptom thereof. For example, treatment can include diminishment of a symptom of a disorder or complete eradication of a disorder. As another example, treatment can include slowing the progression of a disease, or preventing or delaying its recurrence, such as maintenance treatment to prevent or delay relapse.

As used herein, the term “pharmaceutical composition” refers to the combination of an active agent with an excipient, inert or active, making the composition suitable for administration to a human being.

The phrase “pharmaceutically acceptable” is employed herein to refer to those compounds, materials, compositions, and/or dose forms which are, within the scope of sound medical judgment, suitable for use in contact with the tissues of human beings and animals with acceptable toxicity, irritation, allergic response, and other problems or complications commensurate with a reasonable benefit/risk ratio.

As used herein, the term “pharmaceutically acceptable excipient” refers to any of the standard pharmaceutical excipients suitable for use in human beings. For examples of excipients, see e.g., Martin, Remington's Pharmaceutical Sciences, 15th Ed., Mack Publ. Co., Easton, Pa. [1975].

As used herein, the term “pharmaceutically acceptable salt” refers to any salt (e.g., acid or base) of a compound of the present invention which is suitable for administration to a human being. “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Examples of bases include, but are not limited to, alkali metals (e.g., sodium) hydroxides, alkaline earth metals (e.g., magnesium), hydroxides, ammonia, and compounds of formula NR₃, wherein R is C₁₋₄ alkyl, and the like.

Further examples of salts include ion pairs made using the ion pairing agents described in U.S. Pat. No. 8,263,653, the entire disclosure of which is incorporated by reference herein. Still further ion pairing agents can be selected with guidance from Handbook of Pharmaceutical Salts Properties, Selection and Use, UIPAC, Wiley-VCH, P. H. Stahl, ed., the entire disclosure of which is incorporated by reference herein.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “refractory acute myeloid leukemia” refers to failure to achieve complete remission (CR) or complete remission with incomplete recovery (CRi) following: a) two standard dose cytarabine based induction cycles or one high dose cytarabine (HiDAC) based cycle, b) persistent disease after one cycle of standard dose cytarabine (defined as no decrease in marrow blast percentage from diagnosis on Day 14 marrow), or c) persistent disease after at least 2 cycles of a hypomethylating agent with or without venetoclax.

As used herein, the term “relapsed acute myeloid leukemia” refers to the development of recurrent AML (as described by Darner H et al., 2017, supra) after complete remission (CR) or complete remission with incomplete recovery (CRi) has been achieved with a prior chemotherapy or after disease progression on a hypomethylating agent with or without venetoclax.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited steps.

As a general matter, compositions specifying a percentage are by weight unless otherwise specified.

### II. Therapeutic Applications

The invention provides a method for treating acute myeloid leukemia. The method comprises administering to a patient in need thereof devimistat, cytarabine, and mitoxantrone pursuant to an induction cycle of 14 days, wherein during the induction cycle the devimistat is administered as a single daily dose of about 2,000 mg/m² on each of days 1, 2, 3, 4, and 5, the cytarabine is administered in five doses of about 1.0 g/m² each every 12 hours beginning on day 3, and the mitoxantrone is administered as a single daily dose of about 6 mg/m² on each of days 3, 4, and 5, in order to treat the acute myeloid leukemia. The method may be further characterized according to one or more features described herein.

In preferred embodiments, the invention provides a method for treating relapsed or refractory acute myeloid leukemia in a patient who is at least 50 years old, comprising the step of administering to the patient devimistat, cytarabine, and mitoxantrone pursuant to an induction cycle of 14 days, wherein during the induction cycle the devimistat is administered as a single daily dose of about 2,000 mg/m² on each of days 1, 2, 3, 4, and 5, the cytarabine is administered in five doses of about 1.0 g/m² each every 12 hours beginning on day 3, and the mitoxantrone is administered as a single daily dose of about 6 mg/m² on each of days 3, 4, and 5, in order to treat the relapsed or refractory acute myeloid leukemia.

### Type of Acute Myeloid Leukemia

The method may be further characterized according to the severity or type of acute myeloid leukemia. In certain embodiments, the acute myeloid leukemia is refractory to prior standard therapy. In certain embodiments, the acute myeloid leukemia is relapsed from prior standard therapy. Preferably, the acute myeloid leukemia is relapsed or refractory.

### Devimistat

In the present invention devimistat may be administered in any suitable form, including as a solid or liquid, a free acid or salt. In certain embodiments, the devimistat is administered to the patient as a salt or ion pair. In certain embodiments, the devimistat is administered to the patient as a salt or ion pair with triethanolamine.

The devimistat may be formulated in a pharmaceutical composition comprising a pharmaceutically acceptable excipient. In certain embodiments, the pharmaceutical composition comprises devimistat and a pharmaceutically acceptable excipient. In certain embodiments, the pharmaceutical composition comprises an ion pair of devimistat and a pharmaceutically acceptable excipient. In certain embodiments, the pharmaceutical composition comprises a pharmaceutically acceptable salt of devimistat and a pharmaceutically acceptable excipient. In certain embodiments, the pharmaceutical composition comprises devimistat and triethanolamine. In certain embodiments, the pharmaceutical composition comprises devimistat in the form of an ion pair with triethanolamine. In certain embodiments, the pharmaceutical composition further comprises dextrose and water.

In certain embodiments, the pharmaceutical composition comprises triethanolamine and devimistat in a mole ratio of triethanolamine to devimistat of about 10:1 to about 1:10. In certain embodiments, the mole ratio of triethanolamine to devimistat is about 10:1 to about 5:1. In certain embodiments, the mole ratio of triethanolamine to devimistat is about 8:1.

In certain embodiments, the pharmaceutical composition comprises a 50 mg/mL solution of devimistat in 1M aqueous triethanolamine. In certain embodiments, the pharmaceutical composition comprises a solution of devimistat in 1M aqueous triethanolamine diluted from 50 mg/mL to as low as 12.5 mg/mL with sterile aqueous 5% dextrose for injection (D5W). In certain embodiments, the pharmaceutical composition comprises a solution of devimistat in 1M aqueous triethanolamine diluted from 50 mg/mL to about 12.5 mg/mL with sterile aqueous 5% dextrose for injection (D5W).

Preferably, the pharmaceutical composition is prepared by diluting one mL of a 50 mg/mL solution of devimistat in 1M triethanolamine with 3 mL D5W. Preferably, the diluted solution has a pH of about 8.4 to about 8.8. Preferably, the diluted solution is administered to the patient within 24 hours.

Exemplary ion pairing agents that may be used include, for example, a tertiary amine (such as triethanolamine), other amines such as diethanolamine, monoethanolamine, mefenamic acid and tromethamine, and combinations thereof. In certain embodiments, the ion pairing agent is an organic Bronsted base. In certain other embodiments, the ion pairing agent is an amine compound. In yet other embodiments, the ion pairing agent is a monoalkylamine, dialkylamine, trialkylamine, amino-substituted aliphatic alcohol, hydroxymonoalkylamine, hydroxydialkylamine, hydroxytrialkylamine, amino-substituted heteroaliphatic alcohol, alkyldiamine, substituted alkyldiamine, or optionally substituted heteroaryl group containing at least one ring nitrogen atom.

Additional exemplary ion pairing agents include, for example, polyethyleneimine, polyglutamic acid, ammonia, L-arginine, benethamine benzathine, betaine, calcium hydroxide, choline, deanol, diethanolamine(2,2′-iminobis(ethanol)), diethylamine, 2-(diethylamino)-ethanol, ethanolamine, ethylenediamine, N-methyl-glucamine, hydrabamine, 1H-imidazole, lysine, magnesium hydroxide, 4-(2-hydroxyethyl)-morpholine, piperazine, potassium hydroxide, 1-(2-hydroxyethyl)-pyrrolidine, sodium hydroxide, triethanolamine (2,2′,2″-nitrilotris(ethanol)), tromethamine, and zinc hydroxide. In certain other embodiments, the ion pairing agent is diisopropanolamine, 3-amino-1-propanol, meglumine, morpholine, pyridine, niacinamide, tris(hydroxymethyl)aminomethane, 2-((2-dimethylamino)ethoxy)ethanol, 2-(dimethylamino)ethanol, 1-(2-hydroxyethyl)pyrrolidine, or ammonium hydroxide. In certain other embodiments, the ion pairing agent is an alkali metal hydroxide or alkaline earth metal hydroxide, such as, for example, cesium hydroxide.

### Exemplary Route of Administration

The therapeutic method may be further characterized according to the route of administration. For example, in certain embodiments, the devimistat is administered intravenously to the patient. In certain embodiments, the devimistat is administered as an IV infusion over two hours. In certain embodiments, the devimistat is administered as an IV infusion over two hours via a central venous catheter. Preferably, a pharmaceutical composition prepared by diluting a 50 mg/mL solution of devimistat in 1M aqueous triethanolamine to 12.5 mg/mL with sterile aqueous 5% dextrose for injection (D5W) is administered over 2 hours, concurrently with D5W at the rate of about 125 mL/hour, by IV infusion via a central line catheter. More preferably, a pharmaceutical composition prepared by diluting a 50 mg/mL solution of devimistat in 1M aqueous triethanolamine to 12.5 mg/mL with sterile aqueous 5% dextrose for injection (D5W) (i.e., 1 mL of the 50 mg/mL solution is combined with 3 mL of D5W) is administered over 2 hours, concurrently with D5W at the rate of about 125 mL/hour, by IV infusion via a central line catheter, using an infusion pump, that is free flowing and free of air the dead space of the IV catheter. Preferably, the IV line is flushed with D5W after administration of devimistat. Preferably, the IV line is flushed with about 10 mL of D5W after administration of devimistat. Preferably, DEHP-containing IV infusion sets, IV bags, and syringes are not used for mixing or administration of devimistat.

### Exemplary Dosing Amounts

The therapeutic method may be further characterized according to the dose of the devimistat administered to the patient. In certain embodiments, the devimistat is administered at a dose of about 2,500 mg/m² or less on any day it is administered to the patient. In certain embodiments, the devimistat is administered at a dose of about 2,500 mg/m² on any day it is administered to the patient. In certain embodiments, the devimistat is administered at a dose of about 2,000 mg/m² or less on any day it is administered to the patient. In certain embodiments, the devimistat is administered at a dose of about 2,000 mg/m² on any day it is administered to the patient. In certain embodiments, the devimistat is administered at a dose of about 1,875 mg/m² or less on any day it is administered to the patient. In certain embodiments, the devimistat is administered at a dose of about 1,875 mg/m² on any day it is administered to the patient. In certain embodiments, the devimistat is administered at a dose of about 1,500 mg/m² or less on any day it is administered to the patient. In certain embodiments, the devimistat is administered at a dose of about 1,500 mg/m² on any day it is administered to the patient. In certain embodiments, the devimistat is administered at a dose of about 1,000 mg/m² or less on any day it is administered to the patient. In certain embodiments, the devimistat is administered at a dose of about 1,250 mg/m² or less on any day it is administered to the patient. In certain embodiments, the devimistat is administered at a dose of about 1,250 mg/m² on any day it is administered to the patient. In certain embodiments, the devimistat is administered at a dose of about 1,000 mg/m² on any day it is administered to the patient. In certain embodiments, devimistat doses of 1,000 mg/m² (i.e., 50% reduction of 2,000 mg/m² dose), 1,250 mg/m² (i.e., 50% reduction of 2,500 mg/m² dose), 1,500 mg/m² (i.e., 25% reduction of 2,000 mg/m² dose), or 1,875 mg/m² (i.e., 25% reduction of 2,500 mg/m² dose) are administered if the patient experiences a toxicity possibly related to devimistat, such as a grade 2, 3, or 4 toxicity described in Example 1.

### Cytarabine

In the present invention cytarabine may be administered in any suitable form. In certain embodiments, an aqueous solution of cytarabine is administered intravenously to the patient. In certain embodiments, an aqueous solution of cytarabine is administered to the patient by IV infusion over 3 hours.

Any suitable formulation of cytarabine may be used. Cytarabine is commercially available in several pharmaceutically acceptable formulations. In certain embodiments, cytarabine is obtained as a 20 mg/mL or 100 mg/mL solution in sterile water for injection. In certain embodiments, cytarabine is obtained as a 100 mg/mL solution in sterile water for injection. In certain embodiments, cytarabine is obtained as a solution of 2 g cytarabine in 20 mL sterile water for injection (100 mg/mL). In certain embodiments, cytarabine is obtained in as a sterile powder in a 100 mg, 500 mg, 1 g, or 2 g vial for reconstitution with water for injection. In certain embodiments, the cytarabine is obtained as a 100 mg/mL solution in sterile water for injection. In certain embodiments, the cytarabine is obtained as a sterile powder in a 1 g vial and is reconstituted with 10 mL water for injection. In certain embodiments, the cytarabine is obtained as a sterile powder in a 1 g vial and is reconstituted with 10 mL bacteriostatic water for injection with benzyl alcohol 0.945% w/v added as a preservative. In certain embodiments, the cytarabine is obtained as a sterile powder in a 2 g vial and is reconstituted with 20 mL water for injection. In certain embodiments, the cytarabine is obtained as a sterile powder in a 2 g vial and is reconstituted with 20 mL bacteriostatic water for injection with benzyl alcohol 0.945% w/v added as a preservative

### Exemplary Dosing Amounts

The therapeutic method may be further characterized according to the dose of the cytarabine administered to the patient. In certain embodiments, the cytarabine is administered at a dose of about 1 g/m² or less. In certain embodiments, the cytarabine is administered at a dose of about 1 g/m² or less every 12 hours for 5 total doses. In certain embodiments, the cytarabine is administered at a dose of about 1 g/m² or less every 12 hours for 3 total doses. In certain embodiments, the cytarabine is administered at a dose of about 1 g/m². In certain embodiments, the cytarabine is administered at a dose of about 1 g/m² every 12 hours for 5 total doses. In certain embodiments, the cytarabine is administered at a dose of about 1 g/m² every 12 hours for 3 total doses. In certain embodiments, the cytarabine is administered at a dose of about 750 mg/m² or less. In certain embodiments, the cytarabine is administered at a dose of about 750 mg/m² or less every 12 hours for 5 total doses. In certain embodiments, the cytarabine is administered at a dose of about 750 mg/m² or less every 12 hours for 3 total doses. In certain embodiments, the cytarabine is administered at a dose of about 750 mg/m². In certain embodiments, the cytarabine is administered at a dose of about 750 mg/m² every 12 hours for 5 total doses. In certain embodiments, the cytarabine is administered at a dose of about 750 mg/m² every 12 hours for 3 total doses. In certain embodiments, the cytarabine is administered at a dose of about 500 mg/m² or less. In certain embodiments, the cytarabine is administered at a dose of about 500 mg/m² or less every 12 hours for 5 total doses. In certain embodiments, the cytarabine is administered at a dose of about 500 mg/m² or less every 12 hours for 3 total doses. In certain embodiments, the cytarabine is administered at a dose of about 500 mg/m². In certain embodiments, the cytarabine is administered at a dose of about 500 mg/m² every 12 hours for 5 total doses. In certain embodiments, the cytarabine is administered at a dose of about 500 mg/m² every 12 hours for 3 total doses. In certain embodiments, cytarabine doses of 750 mg/m² (i.e., 25% reduction of 1 g/m² dose) or 500 mg/m² (i.e., 50% reduction of 1 g/m² dose) are administered if the patient experiences a toxicity possibly related to cytarabine, such as a bilirubin level of 1.5-3.0 mg/dL or >3.0 mg/dL, or a serum creatinine clearance <60 mL/min per Cockcroft Gault (CG) formula or <60 mL/min/1.73 m² per modified diet in renal disease (MDRD) estimated glomerular filtration rate (eGFR) formula as described in Example 1.

### Mitoxantrone

In the present invention mitoxantrone may be administered in any suitable form. In certain embodiments, an aqueous solution of mitoxantrone hydrochloride is administered intravenously to the patient. In certain embodiments, an aqueous solution of mitoxantrone hydrochloride is administered to the patient by IV infusion over 15 minutes.

Any suitable formulation of mitoxantrone may be used. Mitoxantrone is commercially available in several pharmaceutically acceptable formulations. In certain embodiments, mitoxantrone is obtained as an aqueous solution containing mitoxantrone hydrochloride equivalent to 2 mg/mL free base. In certain embodiments, mitoxantrone is obtained in a vial containing mitoxantrone hydrochloride equivalent to 20 mg mitoxantrone free base in a 10 mL aqueous solution (2 mg/mL). In certain embodiments, mitoxantrone is obtained in a vial containing mitoxantrone hydrochloride equivalent to 25 mg mitoxantrone free base in a 12.5 mL aqueous solution (2 mg/mL). In certain embodiments, mitoxantrone is obtained in a vial containing mitoxantrone hydrochloride equivalent to 30 mg mitoxantrone free base in a 15 mL aqueous solution (2 mg/mL).

### Exemplary Dosing Amounts

The therapeutic method may be further characterized according to the dose of the mitoxantrone administered to the patient. In certain embodiments, the mitoxantrone is administered at a dose of about 6 mg/m² or less on any day it is administered to the patient. In certain embodiments, the mitoxantrone is administered at a dose of about 6 mg/m² on any day it is administered to the patient. In certain embodiments, the mitoxantrone is administered at a dose of about 4.5 mg/m² or less on any day it is administered to the patient. In certain embodiments, the mitoxantrone is administered at a dose of about 4.5 mg/m² on any day it is administered to the patient. In certain embodiments, a mitoxantrone dose of 4.5 mg/m² (i.e., 25% reduction of 6 mg/m² dose) is administered if the patient experiences a toxicity possibly related to mitoxantrone, such as a bilirubin level of >3 mg/dL as described in Example 1.

### Exemplary Treatment Schedule

First Induction Cycle

The method comprises administering to a patient in need thereof devimistat, cytarabine, and mitoxantrone pursuant to an induction cycle of 14 days, wherein during the induction cycle the devimistat is administered as a single daily dose on each of days 1, 2, 3, 4, and 5, the cytarabine is administered in five doses every 12 hours beginning on day 3, and the mitoxantrone is administered as a single daily dose on each of days 3, 4, and 5. In certain embodiments, the devimistat is administered as a single daily dose of about 2,000 mg/m² on each of days 1, 2, 3, 4, and 5, the cytarabine is administered in five doses of about 1.0 g/m² each every 12 hours beginning on day 3, and the mitoxantrone is administered as a single daily dose of about 6 mg/m² on each of days 3, 4, and 5. In certain embodiments, the method comprises administering to a patient with relapsed or refractory AML devimistat, cytarabine, and mitoxantrone pursuant to an induction cycle of 14 days, wherein during the induction cycle the devimistat is administered as a single daily dose of about 2,000 mg/m² on each of days 1, 2, 3, 4, and 5 (total of 5 doses), each devimistat dose being administered over 2 hours as a central line IV infusion, the cytarabine is administered in five doses of about 1.0 g/m² each every 12 hours beginning on day 3 after completion of the devimistat infusion, each cytarabine dose being administered over 3 hours as a central line IV infusion, and the mitoxantrone is administered as a single daily dose of about 6 mg/m² on each of days 3, 4, and 5 after completion of the cytarabine infusion, each mitoxantrone dose being administered over 15 minutes as a central line IV infusion. Preferably, the first, third, and fifth cytarabine doses are started less than or equal to 30 minutes after completion of the day 3, day 4, and day 5 devimistat infusions, and the mitoxantrone doses are started less than or equal to 30 minutes after completing the first, third, and fifth cytarabine infusions. The administered dose of any of the three agents may be reduced if toxicity is observed as described herein. In certain embodiments, during the first induction cycle devimistat, cytarabine, and mitoxantrone are administered only on the days set forth herein. In certain embodiments, during the first induction cycle devimistat, cytarabine, and mitoxantrone are administered only at the doses and on the days set forth herein. In certain embodiments, during the first induction cycle devimistat, cytarabine, and mitoxantrone are administered only at the doses and on the days set forth herein, and the patient is administered no other chemotherapy agent for AML. In certain embodiments, during the first induction cycle the devimistat, cytarabine, and mitoxantrone are administered only at the doses and on the days set forth herein, and the patient is administered no other chemotherapy agent for AML during or after the first induction cycle.

Second Induction Cycle

In certain embodiments, the method further comprises, in addition to a first induction cycle, a second induction cycle. The second induction cycle is an optional cycle, based on the patient's bone marrow results (blasts) at the end of the first induction cycle, preferably on day 14 of the first induction cycle. Preferably, if a second induction cycle is performed it begins than or equal to five calendar days following day 14 of the first induction cycle. In certain embodiments, if the patient's bone marrow results indicate no residual disease, or if no day 14 bone marrow aspirate/biopsy is obtained, the patent will not be treated with a second induction cycle. Preferably, if the patient's day 14 bone marrow aspirate/biopsy result shows less than 5% myeloblasts, or if no day 14 bone marrow aspirate/biopsy is obtained, the patent will not be treated with a second induction cycle. In certain embodiments, the second induction cycle is identical to the first induction cycle. In certain embodiments, if the patient's bone marrow results indicate significant residual disease, the patient will be treated with a second induction cycle identical to the first induction cycle. Preferably, if the patient's day 14 bone marrow aspirate/biopsy result shows greater than or equal to 30% myeloblasts, or greater than or equal to 5% myeloblasts and cellularity greater than 20%, the patent will be treated with a full induction cycle 2 identical to induction cycle 1. Preferably, the patient is hemodynamically stable (i.e., mean arterial pressure (MAP) greater than 60 mm Hg (not on pressors or fluid boluses), and has maintained an ejection fraction (EF) sufficient to allow treatment with mitoxantrone. Preferably, if the patient's day 14 bone marrow aspirate/biopsy result shows greater than or equal to 5% and less than 30% myeloblasts, and cellularity less than or equal to 20%, the patent will be treated with an abbreviated induction cycle 2. In certain embodiments, the dose of one or more of the administered agents is reduced as described herein, but the second induction cycle is otherwise the same as the first induction cycle. In certain embodiments, the second induction cycle is an abbreviated cycle of 14 days, wherein during the abbreviated induction cycle the devimistat is administered as a single daily dose on each of days 1, 2, and 3, the cytarabine is administered in three doses every 12 hours beginning on day 2, and the mitoxantrone is administered as a single daily dose on each of days 2 and 3. In certain embodiments, the second induction cycle is an abbreviated cycle of 14 days, wherein during the abbreviated induction cycle the devimistat is administered as a single daily dose of about 2,000 mg/m² on each of days 1, 2, and 3, the cytarabine is administered in three doses of about 1.0 g/m² each every 12 hours beginning on day 2, and the mitoxantrone is administered as a single daily dose of about 6 mg/m² on each of days 2 and 3. In certain embodiments, if the patient's bone marrow results indicate minimal residual disease, the patient will be treated with a second induction cycle that is an abbreviated cycle. In certain embodiments, the method further comprises, after treating a patient who is at least 50 years old with relapsed or refractory AML with a first induction cycle as described above, administering to the patient devimistat, cytarabine, and mitoxantrone pursuant to an abbreviated induction cycle 2 of 14 days, wherein during the abbreviated induction cycle 2 the devimistat is administered as a single daily dose of about 2,000 mg/m² on each of days 1, 2, and 3 (total of 3 doses), each devimistat dose being administered over 2 hours as a central line IV infusion, the cytarabine is administered in three doses of about 1.0 g/m² each every 12 hours beginning on day 2 after completion of the devimistat infusion, each cytarabine dose being administered over 3 hours as a central line IV infusion, and the mitoxantrone is administered as a single daily dose of about 6 mg/m² on each of days 2 and 3 after completion of the cytarabine infusion, each mitoxantrone dose being administered over 15 minutes as a central line IV infusion. Preferably, the first and third cytarabine doses are started less than or equal to 30 minutes after completion of the day 2 and day 3 devimistat infusions, and the mitoxantrone doses are started less than or equal to 30 minutes after completing the first and third cytarabine infusions. Preferably, on days 4 through 14 of the abbreviated induction cycle 2, no devimistat, cytarabine, or mitoxantrone is administered. The administered dose of any of the three agents may be reduced if toxicity is observed as described herein. In certain embodiments, during the second induction cycle devimistat, cytarabine, and mitoxantrone are administered only on the days set forth herein. In certain embodiments, during the second induction cycle devimistat, cytarabine, and mitoxantrone are administered only at the doses and on the days set forth herein. In certain embodiments, during the second induction cycle devimistat, cytarabine, and mitoxantrone are administered only at the doses and on the days set forth herein, and the patient is administered no other chemotherapy agent for AML. In certain embodiments, during the second induction cycle devimistat, cytarabine, and mitoxantrone are administered only at the doses and on the days set forth herein, and the patient is administered no other chemotherapy agent for AML during or after the second induction cycle.

Consolidation Cycle(s)

In certain embodiments, the method further comprises, in addition to a first induction cycle and optionally a second induction cycle (full or abbreviated), one or two consolidation cycles. In certain embodiments, a patient who achieves complete remission (CR) or complete remission with incomplete recovery (CRi) may receive one or two consolidation cycles. In certain embodiments, the patient should also, in the opinion of the treating physician, be eligible for additional therapy. In certain embodiments, a patient is eligible for additional therapy if the patient is tolerating the treatment without undue complications. Preferably, if the patient a) achieves CR or CRi after the first induction cycle or after the first induction cycle and full or abbreviated second induction cycle, b) continues to meet the same organ function and performance status eligibility requirements (re-assessed prior to day 1 of each cycle), and c) for whom allogeneic transplant is not possible or will be delayed by more than 6 weeks, the patient is treated with consolidation therapy. In certain embodiments, the first consolidation cycle will start within three weeks of documentation of CR or CRi. Preferably, the consolidation therapy starts less than or equal to three weeks after the establishment of CR or CRi and is identical to the abbreviated induction cycle 2. In certain embodiments, the patient receives no consolidation cycles. In certain embodiments, the patient receives one consolidation cycle. In certain embodiments, the patient receives two consolidation cycles. In certain embodiments, the second consolidation cycle is started upon count recovery. In certain embodiments, the second consolidation cycle is started upon count recovery in the absence of disease progression. In certain embodiments, the second consolidation cycle is started upon count recover in the absence of disease progression or unacceptable toxicity. In certain embodiments, each consolidation cycle is identical to the abbreviated induction cycle, i.e., a cycle of 14 days, wherein during the consolidation cycle the devimistat is administered as a single daily dose on each of days 1, 2, and 3, the cytarabine is administered in three doses every 12 hours beginning on day 2, and the mitoxantrone is administered as a single daily dose on each of days 2 and 3. In certain embodiments, each consolidation cycle is a cycle of 14 days, wherein during the consolidation cycle the devimistat is administered as a single daily dose of about 2,000 mg/m² on each of days 1, 2, and 3, the cytarabine is administered in three doses of about 1.0 g/m² each every 12 hours beginning on day 2, and the mitoxantrone is administered as a single daily dose of about 6 mg/m² on each of days 2 and 3. In certain embodiments, the method further comprises, after treating a patient who is at least 50 years old with relapsed or refractory AML with a first induction cycle as described above and optionally a full or abbreviated second induction cycle as described above, administering to the patient devimistat, cytarabine, and mitoxantrone pursuant to one or two consolidation cycles of 14 days, wherein during each consolidation cycle the devimistat is administered as a single daily dose of about 2,000 mg/m² on each of days 1, 2, and 3 (total of 3 doses), each devimistat dose being administered over 2 hours as a central line IV infusion, the cytarabine is administered in three doses of about 1.0 g/m² each every 12 hours beginning on day 2 after completion of the devimistat infusion, each cytarabine dose being administered over 3 hours as a central line IV infusion, and the mitoxantrone is administered as a single daily dose of about 6 mg/m² on each of days 2 and 3 after completion of the cytarabine infusion, each mitoxantrone dose being administered over 15 minutes as a central line IV infusion. Preferably, the first and third cytarabine doses are started less than or equal to 30 minutes after completion of the day 2 and day 3 devimistat infusions, and the mitoxantrone doses are started less than or equal to 30 minutes after completing the first and third cytarabine infusions. Preferably, on days 4 through 14 of each consolidation cycle, no devimistat, cytarabine, or mitoxantrone is administered. The administered dose of any of the three agents may be reduced if toxicity is observed as described herein. In certain embodiments, during each consolidation cycle devimistat, cytarabine, and mitoxantrone are administered only on the days set forth herein. In certain embodiments, during each consolidation cycle devimistat, cytarabine, and mitoxantrone are administered only at the doses and on the days set forth herein. In certain embodiments, during each consolidation cycle devimistat, cytarabine, and mitoxantrone are administered only at the doses and on the days set forth herein, and the patient is administered no other chemotherapy agent for AML. In certain embodiments, during each consolidation cycle devimistat, cytarabine, and mitoxantrone are administered only at the doses and on the days set forth herein, and the patient is administered no other chemotherapy agent for AML during or after completing the consolidation cycle(s).

Maintenance Cycle(s)

In certain embodiments, the method further comprises, in addition to a first induction cycle, optionally a second induction cycle, and optionally one or two consolidation cycles, one or more maintenance cycles. In certain embodiments, a patient who achieves CR or CRi according to standard response criteria for AML (Döhner H et al. 2017, supra) and has completed 0, 1, or 2 consolidation cycles may receive at least one maintenance cycle. In certain embodiments, a patient who achieves CR or CRi according to standard response criteria for AML (Döhner H et al. 2017, supra), has completed 0, 1, or 2 consolidation cycles, and is not eligible for or refuses hematopoietic stem cell transplantation (HSCT), or for whom there will be a delay prior to HSCT, may receive at least one maintenance cycle. Preferably, if the patient a) achieves CR or CRi after the first induction cycle, the full or abbreviated second induction cycle, or the first or second consolidation cycle, and b) is not eligible for or refuses hematopoietic stem cell transplantation (HSCT), or for whom there will be a delay prior to HSCT of greater than or equal to 6 weeks, the patient will be treated with maintenance therapy. In certain embodiments, maintenance cycles may be repeated until relapse, availability of HSCT, or development of unacceptable toxicity. In certain embodiments, the patient receives no maintenance cycles. In certain embodiments, the patient receives one maintenance cycle. In certain embodiments, the patient receives two maintenance cycles. In certain embodiments, the patient receives more than two maintenance cycles. In certain embodiments, the patient receives at least five maintenance cycles. In certain embodiments, the patient receives at least ten maintenance cycles. In certain embodiments, each maintenance cycle is a cycle of 28 days, wherein during the maintenance cycle devimistat is administered as a single daily dose on each of days 1, 2, 3, 4, and 5. In certain embodiments, each maintenance cycle is a cycle of 28 days, wherein during the maintenance cycle devimistat is administered as a single daily dose of about 2,500 mg/m² on each of days 1, 2, 3, 4, and 5. In certain embodiments, cytarabine and mitoxantrone are not administered during the maintenance cycle. In certain embodiments, each maintenance cycle is a cycle of 28 days, wherein during the maintenance cycle devimistat is administered as a single daily dose on each of days 1, 2, 3, 4, and 5, and cytarabine and mitoxantrone are not administered. In certain embodiments, each maintenance cycle is a cycle of 28 days, wherein during the maintenance cycle devimistat is administered as a single daily dose of about 2,500 mg/m² on each of days 1, 2, 3, 4, and 5, and cytarabine and mitoxantrone are not administered. In certain embodiments, the method further comprises, after treating a patient who is at least 50 years old with relapsed or refractory AML with a first induction cycle as described above, optionally a full or abbreviated second induction cycle as described above, and optionally one or two consolidation cycles as described above, administering to the patient devimistat pursuant to one or more maintenance cycles of 28 days, wherein during each maintenance cycle the devimistat is administered as a single daily dose of about 2,500 mg/m² on each of days 1, 2, 3, 4, and 5 (total of 5 doses), each devimistat dose being administered over 2 hours as a central line IV infusion, and no devimistat is administered on days 6 through 28. The administered dose of devimistat may be reduced if toxicity is observed as described herein. In certain embodiments, during each maintenance cycle devimistat is administered only on the days set forth herein. In certain embodiments, during each maintenance cycle devimistat is administered only at the doses and on the days set forth herein. In certain embodiments, during each maintenance cycle devimistat is administered only at the doses and on the days set forth herein, and the patient is administered no other chemotherapy agent for AML. In certain embodiments, during each maintenance cycle devimistat is administered only at the doses and on the days set forth herein, and the patient is administered no other chemotherapy agent for AML during or after completing the maintenance cycle(s).

### Treatment Efficacy and Safety

The therapeutic method of the present invention may be further characterized by the efficacy and safety of the treatment. Preferably, the method provides an acceptable safety profile, with the benefit of treatment outweighing the risk. When tested in a phase III clinical trial of at least 50 patients ≥50 years old with relapsed or refractory acute myeloid leukemia, the method of the present invention preferably provides a complete remission (CR) rate of at least 10%, i.e., at least 10% of the patients treated according to the method of the present invention achieve CR. Preferably, the phase III clinical trial comprises at least 60 patients. More preferably, the phase III clinical trial comprises at least 70 patients. More preferably, the phase III clinical trial comprises at least 80 patients. More preferably, the phase III clinical trial comprises at least 90 patients. Most preferably, the phase III clinical trial comprises at least 100 patients. Preferably, the phase III clinical trial is conducted according to the procedure set forth in Example 1. Preferably, the CR rate is at least 20%. More preferably, the CR rate is at least 30%. More preferably, the CR rate is at least 40%. More preferably, the CR rate is at least 50%. More preferably, the CR rate is at least 60%. More preferably, the CR rate is at least 70%. More preferably, the CR rate is at least 80%. More preferably, the CR rate is at least 90%. Preferably, the CR rate is significantly higher than the control (HAM) arm, i.e., the CR rate of the patients in the phase III clinical trial treated according to the method of the present invention is significantly higher than the CR rate of the patients treated in the control (HAM) arm of the clinical trial. Preferably, the CR rate is at least 5 percentage points higher than the control arm (e.g., if the CR rate of the patients in the clinical trial treated according to the method of the present invention is 10%, 35%, or 73%, then the CR rate of the control arm patients is ≤5%, ≤30%, or ≤68%, respectively). More preferably, the CR rate is at least 10 percentage points higher than the control arm. More preferably, the CR rate is at least 15 percentage points higher than the control arm. More preferably, the CR rate is at least 20 percentage points higher than the control arm. More preferably, the CR rate is at least 25 percentage points higher than the control arm. More preferably, the CR rate is at least 30 percentage points higher than the control arm. More preferably, the CR rate is at least 35 percentage points higher than the control arm. More preferably, the CR rate is at least 40 percentage points higher than the control arm. More preferably, the CR rate is at least 45 percentage points higher than the control arm. More preferably, the CR rate is at least 50 percentage points higher than the control arm. More preferably, the CR rate is at least 55 percentage points higher than the control arm. More preferably, the CR rate is at least 60 percentage points higher than the control arm. More preferably, the CR rate is at least 65 percentage points higher than the control arm. More preferably, the CR rate is at least 70 percentage points higher than the control arm. More preferably, the CR rate is at least 75 percentage points higher than the control arm. More preferably, the CR rate is at least 80 percentage points higher than the control arm. More preferably, the CR rate is at least 85 percentage points higher than the control arm.

Preferably, when tested in a phase III clinical trial as described herein the method of the present invention provides an overall survival (OS) of at least 3 months. More preferably, the OS is at least 4 months. More preferably, the OS is at least 5 months. More preferably, the OS is at least 6 months. More preferably, the OS is at least 7 months. More preferably, the OS is at least 8 months. More preferably, the OS is at least 9 months. More preferably, the OS is at least 10 months. More preferably, the OS is at least 11 months. More preferably, the OS is at least 12 months. More preferably, the OS is at least 13 months. More preferably, the OS is at least 14 months. More preferably, the OS is at least 15 months. More preferably, the OS is at least 16 months. More preferably, the OS is at least 17 months. More preferably, the OS is at least 18 months. More preferably, the OS is at least 19 months. More preferably, the OS is at least 20 months. More preferably, the OS is at least 21 months. More preferably, the OS is at least 22 months. More preferably, the OS is at least 23 months. More preferably, the OS is at least 24 months. Preferably, the OS is significantly higher than the control (HAM) arm, i.e., the OS of the patients in the phase III clinical trial treated according to the method of the present invention is significantly higher than the OS of the patients treated in the control (HAM) arm of the clinical trial. Preferably, the OS is at least 1 month longer than the control arm (e.g., if the OS of the patients in the clinical trial treated according to the method of the present invention is 5 months, 8 months, or 12 months, then the OS of the control arm patients is ≤4 months, ≤7 months, or ≤11 months, respectively). More preferably, the OS is at least 2 months longer than the control arm. More preferably, the OS is at least 3 months longer than the control arm. More preferably, the OS is at least 4 months longer than the control arm. More preferably, the OS is at least 5 months longer than the control arm. More preferably, the OS is at least 6 months longer than the control arm. More preferably, the OS is at least 7 months longer than the control arm. More preferably, the OS is at least 8 months longer than the control arm. More preferably, the OS is at least 9 months longer than the control arm. More preferably, the OS is at least 10 months longer than the control arm. More preferably, the OS is at least 11 months longer than the control arm. More preferably, the OS is at least 12 months longer than the control arm. More preferably, the OS is at least 13 months longer than the control arm. More preferably, the OS is at least 14 months longer than the control arm. More preferably, the OS is at least 15 months longer than the control arm. More preferably, the OS is at least 16 months longer than the control arm. More preferably, the OS is at least 17 months longer than the control arm. More preferably, the OS is at least 18 months longer than the control arm.

Preferably, when tested in a phase III clinical trial as described herein the method of the present invention provides a complete remission+complete remission with partial hematologic recovery (CR+CRh) rate of at least 10%, i.e., at least 10% of the patients treated according to the method of the present invention achieve CR or CRh. Preferably, the CR+CRh rate is at least 20%. More preferably, the CR+CRh rate is at least 30%. More preferably, the CR+CRh rate is at least 40%. More preferably, the CR+CRh rate is at least 50%. More preferably, the CR+CRh rate is at least 60%. More preferably, the CR+CRh rate is at least 70%. More preferably, the CR+CRh rate is at least 80%. More preferably, the CR+CRh rate is at least 90%. Preferably, the CR+CRh rate is significantly higher than the control (HAM) arm, i.e., the CR+CRh rate of the patients in the phase III clinical trial treated according to the method of the present invention is significantly higher than the CR+CRh rate of the patients treated in the control (HAM) arm of the clinical trial. Preferably, the CR+CRh rate is at least 5 percentage points higher than the control arm (e.g., if the CR+CRh rate of the patients in the clinical trial treated according to the method of the present invention is 10%, 35%, or 73%, then the CR+CRh rate of the control arm patients is ≤5%, ≤30%, or ≤68%, respectively). More preferably, the CR+CRh rate is at least 10 percentage points higher than the control arm. More preferably, the CR+CRh rate is at least 15 percentage points higher than the control arm. More preferably, the CR+CRh rate is at least 20 percentage points higher than the control arm. More preferably, the CR+CRh rate is at least 25 percentage points higher than the control arm. More preferably, the CR+CRh rate is at least 30 percentage points higher than the control arm. More preferably, the CR+CRh rate is at least 35 percentage points higher than the control arm. More preferably, the CR+CRh rate is at least 40 percentage points higher than the control arm. More preferably, the CR+CRh rate is at least 45 percentage points higher than the control arm. More preferably, the CR+CRh rate is at least 50 percentage points higher than the control arm. More preferably, the CR+CRh rate is at least 55 percentage points higher than the control arm. More preferably, the CR+CRh rate is at least 60 percentage points higher than the control arm. More preferably, the CR+CRh rate is at least 65 percentage points higher than the control arm. More preferably, the CR+CRh rate is at least 70 percentage points higher than the control arm. More preferably, the CR+CRh rate is at least 75 percentage points higher than the control arm. More preferably, the CR+CRh rate is at least 80 percentage points higher than the control arm. More preferably, the CR+CRh rate is at least 85 percentage points higher than the control arm.

### Patients for Treatment

The therapeutic methods may be further characterized according to the patient to be treated. Preferably, the patient is a human. In certain embodiments, the patient is an adult human. In certain embodiments, the patient is an adult human ≥50 years old. In certain embodiments, the patient is an adult human ≥60 years old.

### III. Medical Kits

Another aspect of the invention provides medical kits containing a therapeutic agent and/or pharmaceutical composition described herein, along with instructions for using the kits to treat acute myeloid leukemia according to the therapeutic applications described herein. In certain embodiments, the medical kit comprises (i) devimistat and (ii) instructions for treating acute myeloid leukemia in a patient using the devimistat according to the therapeutic applications described herein. In certain embodiments, the medical kit comprises (i) devimistat and (ii) instructions for treating acute myeloid leukemia in a patient using a combination of devimistat, cytarabine, and mitoxantrone according to the therapeutic applications described herein.

In certain embodiments, the medical kit comprises (i) devimistat and (ii) instructions for treating acute myeloid leukemia in a patient in need thereof using the devimistat in combination with cytarabine and mitoxantrone pursuant to an induction cycle of 14 days, wherein during the induction cycle the devimistat is administered as a single daily dose of about 2,000 mg/m² on each of days 1, 2, 3, 4, and 5, the cytarabine is administered in five doses of about 1.0 g/m² each every 12 hours beginning on day 3, and the mitoxantrone is administered as a single daily dose of about 6 mg/m² on each of days 3, 4, and 5, in order to treat the acute myeloid leukemia.

### IV. Treatment Methods

Another aspect of the invention provides treatment methods in which devimistat is provided, along with instructions for using it to treat acute myeloid leukemia according to the therapeutic applications described herein. In certain embodiments, the treatment method comprises providing (i) devimistat and (ii) instructions for treating acute myeloid leukemia in a patient using the devimistat according to the therapeutic applications described herein. In certain embodiments, the treatment method comprises providing (i) devimistat and (ii) instructions for treating acute myeloid leukemia in a patient using the devimistat in combination with cytarabine and mitoxantrone according to the therapeutic applications described herein.

In certain embodiments, the treatment method comprises providing (i) devimistat and (ii) instructions for treating acute myeloid leukemia in a patient in need thereof using the devimistat in combination with cytarabine and mitoxantrone pursuant to an induction cycle of 14 days, wherein during the induction cycle the devimistat is administered as a single daily dose of about 2,000 mg/m² on each of days 1, 2, 3, 4, and 5, the cytarabine is administered in five doses of about 1.0 g/m² each every 12 hours beginning on day 3, and the mitoxantrone is administered as a single daily dose of about 6 mg/m² on each of days 3, 4, and 5, in order to treat the acute myeloid leukemia.

The description above describes multiple aspects and embodiments of the invention, including therapeutic applications, treatment methods, pharmaceutical compositions, and medical kits. The patent application specifically contemplates all combinations and permutations of the aspects and embodiments.

### Examples

The invention now being generally described, will be more readily understood by reference to the following examples, which are included merely for purposes of illustration of certain aspects and embodiments of the present invention, and are not intended to limit the invention.

### Example 1—Treatment of Relapsed or Refractory Acute Myeloid Leukemia in Older Human Patients (≥50 Years) Using Devimistat in Combination with High Dose Cytarabine and Mitoxantrone (CHAM)

### Study Design

A prospective, multi-center, open label, randomized Phase III study of CHAM compared to HAM in older patients (≥50 years) with relapsed/refractory Acute Myeloid Leukemia. There are two study arms: Arm 1=CHAM (i.e. 6,8-bis-benzylthio-octanoic acid (CPI-613®; devimistat)+High Dose Cytarabine and Mitoxantrone); Arm 2=HAM (i.e. High Dose Cytarabine and Mitoxantrone). The target sample size is 500 patients, 250 patients in each of two arms. Stratification factors: a) prior therapy: HiDAC based vs. hypomethylator (azacytidine or decitabine, HMA) vs. 7+3; b) relapsed vs refractory AML; c) cytogenetic risk category: favorable, intermediate, poor, and not available, if no data available (historical data may be used to define risk factors). Refer also to Table 1; d) age (50-69 vs. ≥70 years old); e) performance score (PS; 0-1 vs. 2); 0 treating institution.

The primary objective is to determine efficacy of CHAM in terms of CR (complete remission) and compare it to HAM [control]. CR will be determined as per standard response criteria for AML (Wilmer H et al. 2017, supra).

Secondary objectives are
(a) Efficacy: to determine efficacy of CHAM in terms of OS (overall survival) and CR+CRh (complete remission+complete remission with partial hematologic recovery) as the two key secondary objectives to compare with HAM [control]. OS and CR+CRh will be determined as per standard response criteria for AML (Wilmer H et al. 2017, supra).
(b) Safety: to assess the safety of CHAM based mainly on the frequency of adverse events based on the Common Terminology Criteria for Adverse Events (CTCAE version 5.0 or later) grade. Adverse events will be coded according to the Medical Dictionary for Regulatory Activities. The safety outcomes will include the occurrence of at least one serious adverse event (SAE), of at least one grade 3/4 adverse event, and of at least one adverse event requiring the discontinuation of study treatment. Electrocardiogram (ECG) QTc intervals and cardiac markers will also be evaluated.
(c) Pharmacokinetics (PK): The goal of pharmacokinetic analysis will be to study Cₘₐₓ (maximum (peak) plasma drug concentration), Cₘᵢₙ (minimum (trough) plasma drug concentration), AUC (area under the plasma concentration-time curve), T_{1/2} (elimination half-life (to be used in one-or noncompartmental model), Tₘₐₓ (time to reach maximum (peak) plasma concentration drug administration), CL (apparent total body clearance of the drug from plasma) and V_{d} (apparent volume of distribution) for both 6,8-bis-benzylthio-octanoic acid (devimistat) and its metabolites CPI-2850 and CPI-1810.
(d) Patient-Reported Outcome (PRO): To evaluate patient reported outcomes among patients receiving CHAM compared to HAM, as administered at the following time points: a. Screening/Baseline, b. Time of recovery marrow, c. Upon completion of consolidation therapy (if applicable), d. Every 3 months after completion of consolidation (if applicable) as follows: i. For CHAM Arm: During the maintenance phase, Long-Term Safety and QOL Follow-up period and End of Treatment Visit and/or End of Study Visit, ii. For HAM Arm: During the Long-Term Safety and QOL Follow-up period, End of Treatment Visit and/or End of Study Visit.
(e) Cancer-associated mutations and/or genetic alterations: Cancer-associated mutations and/or genetic alterations in bone marrow aspirates/biopsies and/or peripheral blood. Up to 10 unstained slides from baseline and time of disease progression biopsies will be collected when available. These slides will be used for immunohistochemistry (IHC) staining for PDKs, PDH, KGDH, SOD2 and CD79a. They will also be used as a source of material for RNA and whole exome sequencing should the bone marrow aspirate/biopsy material be inadequate.

Exploratory objectives are
(a) Gene expression analysis by RNA sequencing for baseline bone marrow aspirate/biopsy samples to validate previously described response signature from the phase I study (CCCWFU 22112) to determine the positive and negative predictive value of the gene expression signature found from studying patients on CCCWFU 22112. A separate examination of bone marrow aspirate/biopsy samples will be performed. Samples will be collected from patients at baseline and time to disease progression for RNA sequencing analysis to determine the positive and negative predictive value of the response signature determined by a similar analysis of patient samples from study CCCWFU 22112. Efficacy and safety analyses per gene mutations will also be assessed (these may include FLT3, IDH1/2, TP53, CEBPα, NPM1, etc.).
(b) PK/PD analyses for dose/exposure-response on efficacy and safety;
(c) Patient survival: 30-day and 60-day mortality after first dose of the study related treatment.

### Patient Inclusion Criteria

Patients must meet all of the following inclusion criteria before enrollment; with the main inclusion criteria being #'s 2-6:
1. Patient has provided an informed consent prior to initiation of any study specific activities/procedures.
2. Males and females age ≥50 years must have histologically documented AML that is relapsed from, or refractory to, prior standard therapies.
3. Refractory is defined as failure to achieve CR or CRi following:
   a. 2 standard dose cytarabine based induction cycles or one high dose cytarabine (HiDAC) based cycle, or
   b. Persistent disease after one cycle of standard dose cytarabine (defined as no decrease in marrow blast percentage from diagnosis on day 14 marrow), or
   c. Persistent disease after at least 2 cycles of a hypomethylating agent (azacytidine or decitabine) with or without venetoclax.
4. Relapse is defined as development of recurrent AML (as described by Darner H et al, 2017, supra) after CR or CRi has been achieved with a prior chemotherapy or after disease progression on a hypomethylating agent with or without venetoclax.
5. Eastern Cooperative Oncology Group (ECOG) performance status (PS) 0-2.
6. Expected survival greater than 3 months.
7. Women of child-bearing potential (i.e., women who are pre-menopausal or <2 years post-menopausal or not surgically sterile) must practice a highly effective method of birth control consistent with local regulations regarding the use of birth control methods. Examples: use of oral, injected or implanted hormonal methods of contraception; placement of an intra uterine device (IUD) or intrauterine system (IUS); male partner sterilization (the vasectomized partner should be the sole partner for that subject); true abstinence during and for 6 months after the last administered dose of CHAM or HAM therapy, and must have a negative serum pregnancy test within 1 week prior to treatment initiation and at 1^{st} day of each cycle and at the end of systemic exposure (Note: Pregnant patients are excluded because the effects of CPI-613® (devimistat) on a fetus are unknown).
8. Fertile men who are sexually active with a woman of childbearing potential and has not had a vasectomy must agree to use a barrier method of birth control e.g., either condom with spermicidal foam/gel/film/cream/suppository or partner with occlusive cap (diaphragm or cervical/vault caps) with spermicidal foam/gel/film/cream/suppository during the study period and up to 6 months after completion of the study screening, unless documentation of infertility exists.
9. Good state of mental health, ability to understand and willingness to sign the informed consent form.
10. No radiotherapy, treatment with cytotoxic chemotherapy, treatment with biologic agents or any anti-cancer therapy within the 1 week prior to treatment with CPI-613® (devimistat). Hydroxyurea and oral tyrosine kinase (FLT3) or IDH1/2 inhibitors being used with Grade ≤2 toxicity can be taken until the day prior to starting study therapy. Previous exposure to a hypomethylating agent either alone or in combination with Venetoclax is allowed. Patients must have fully recovered from the acute, non-hematological, non-infectious toxicities of any prior treatment with cytotoxic drugs, radiotherapy or other anti-cancer modalities with the exception of alopecia (returned to baseline status as noted before most recent treatment). Patients with persisting, non-hematologic, non-infectious toxicities from prior treatment ≤Grade 2 are eligible but must be documented as such.
11. Laboratory values ≤2 weeks before dosing must be:
   a. Adequate hepatic function (aspartate aminotransferase/serum glutamic oxaloacetic transaminase [AST/SGOT]≤5×upper limit of normal [ULN], alanine aminotransferase/serum glutamic oxaloacetic transaminase [ALT/SGPT]≤5×ULN, bilirubin ≤1.5×ULN).
   b. Adequate renal function (serum creatinine clearance ≥60 mL/min per CockCroft-Gault formula).
   c. Adequate coagulation (International Normalized Ratio (INR) must be <1.7 unless on vitamin k antagonist anticoagulation).
12. Left Ventricular Ejection Fraction (LVEF) by transthoracic echocardiogram (TTE) or multigated acquisition scan (MUGA) or cardiac magnetic resonance imaging (MRI)) sufficient to safely administer mitoxantrone as determined by the treating physician.
13. No marked baseline prolongation of QT/QTc interval (e.g., repeated exhibition of a QTc interval >450 ms for male and >470 ms for female patients).
14. No history of additional risk factors for torsade de pointes (e.g., clinically significant heart failure, hypokalemia, immediate family history of Long QT Syndrome).
15. Allow only patients who experienced relapse after 1 year from previous HiDAC treatment or who didn't receive HiDAC previously (Note: This inclusion applies only to South Korea).

### Patient Exclusion Criteria (Main Exclusion Criteria are #'s 1, 6, and 19-23)

1. Patients who have received cytotoxic chemotherapy treatment for their current relapsed or refractory AML. Treatment with hypomethylating agents (decitabine or azacytidine) either alone or in combination with venetoclax are allowed. Targeted therapies including FLT3 or IDH1/2 inhibitors or Hydrea or venetoclax are allowed. Targeted therapies and Hydrea may be taken until the day prior to starting CHAM or HAM therapy.
2. Vulnerable adult and patient whose health conditions does not allow them to give their consent.
3. History or evidence of any other clinically significant disorder, condition or disease (e.g. symptomatic congestive heart failure, unstable angina pectoris, symptomatic myocardial infection, uncontrolled cardiac arrhythmia, pericardial disease or heart failure New York Heart Association Class III or IV), or severe debilitating pulmonary disease, that would potentially increase patients' risk for toxicity and in the opinion of the Investigator, would pose a risk to patient safety or interfere with the study evaluation, procedures or completion.
4. Patients with active Central Nervous System (CNS) involvement (leukemic infiltration, blast in the spinal fluid).
5. Any active uncontrolled bleeding, and any patients with a bleeding diathesis (e.g., active peptic ulcer disease).
6. Female patients who are pregnant or breastfeeding, or planning to become pregnant or breastfeed during treatment and for an additional 6 months after the last dose of CHAM or HAM therapy (the teratogenic potential of CPI-613® (devimistat) is unknown). Female patient of childbearing potential with a positive pregnancy test assessed by a serum pregnancy test at Screening.
7. Women of childbearing potential (i.e., women who are pre-monopausal or <2 years postmenopausal or not surgically sterile) unwilling to practice a highly effective method of birth control consistent with local regulations regarding the use of birth control methods during treatment and for 6 months after completion of CHAM or HAM therapy for AML.
8. Male patients with a pregnant partner who are unwilling to practice abstinence or use a condom during treatment and for 6 months after completion of CHAM or HAM therapy.
9. Male patients unwilling to abstain from donating sperm during treatment and for 6 months after completion of CHAM or HAM therapy with potential highest teratogenic risk.
10. Known hypersensitivity to study treatment drugs or any of the excipient(s) contained in the drug formulation.
11. Life expectancy less than 3 months.
12. Any condition or abnormality which may, in the opinion of the investigator, compromise the safety of patients.
13. Unwilling or unable to follow protocol requirements.
14. Patients with large and recurrent pleural or peritoneal effusions requiring frequent drainage (e.g. weekly).
15. Patients with any amount of clinically significant pericardial effusion that requires drainage.
16. Evidence of ongoing, uncontrolled bacterial, viral or fungal infection.
17. Patients with known human immunodeficiency virus infection.
18. History of other malignancy within the past 5 years, with the following exception(s):
   a. Malignancy treated with curative intent and with no known active disease present for ≥5 years before enrolment and felt to be at low risk for recurrence by the treating physician
   b. Adequately treated non-melanoma skin cancer or lentigo maligna without evidence of recurrent or residual disease
   c. Adequately treated cervical carcinoma in situ without evidence of disease
   d. Prostate cancer Stage 1.
19. Patients receiving any other standard or investigational treatment for AML, or any other investigational agent for any indication within the past 1 week prior to initiation of CPI-613® (devimistat) treatment (the use of Hydrea and/or venetoclax, oral tyrosine kinase inhibitors FLT3 or IDH 1/2 inhibitors is allowed until the day prior to starting CHAM or HAM therapy). Previous exposure to a hypomethylating agent either alone or in combination with venetoclax is allowed.
20. Patients who have received immunotherapy of any type within the past 1 week prior to initiation of CPI-613® (devimistat) treatment.
21. Requirement for immediate palliative treatment of any kind including minor surgery.
22. Patients who have received a chemotherapy regimen with autologous stem cell support (bone marrow transplantation) within 6 months of starting CHAM or HAM therapy.
23. Patients who have had allogenic bone marrow transplantation within the last 6 months. Patients who have had an allogenic transplant more than 6 months ago are eligible provided they have no graft vs host disease.
24. Cytarabine contraindications
   Hypersensitivity to the cytarabine or to any of the excipients of cytarabine injection.
   Anemia, leucopenia and thrombocytopenia of non-malignant aetiology (e.g bone marrow aplasia); unless the clinician feels that such management offers the most hopeful alternative for the patient.
   Degenerative and toxic encephalopathies, especially after the use of methotrexate or treatment with ionizing radiation.
25. Mitoxantrone contraindications
   Mitoxantrone Sterile Concentrate is contraindicated in patients who have demonstrated prior hypersensitivity to mitoxantrone hydrochloride, other anthracyclines or any of its components. Use in patients with profound bone marrow suppression is a relative contraindication depending on the clinical circumstances.

### Concomitant Medications, Treatments, and Procedures

All concomitant medications taken during study participation, such as prescription medications, over the counter medication, non-prescription medications, will be recorded on the Case Report Forms (CRFs). For this protocol, a prescription medication is defined as a medication that can be prescribed only by a properly authorized/licensed clinician. The following medications are prohibited:
Previous cytotoxic chemotherapy treatment for relapsed or refractory AML.
Other standard or investigational treatment for AML within the last 1 week (except hydroxyurea, FLT3 and IDH inhibitors) prior to initiation of CPI-613® (devimistat) treatment.
Immunotherapy agents within the 1 week prior to initiation of CPI-613® (devimistat) treatment.

The following medications are permitted: Previous treatment for relapsed or refractory AML with hypomethylating agents with or without venetoclax is permitted. Targeted therapies including FLT3 or IDH1/2 inhibitors or Hydrea are allowed until the day prior to starting CHAM or HAM therapy.

### Prophylactic Medications, Treatments, and Procedures

Patients will receive standard prophylactic treatment for drug-related symptoms. Prophylactic antimicrobials and tumor lysis prophylaxis will be administered as per institutional guidelines. All supportive measures are at the discretion of the Investigator. Supportive treatment may include anti-emetic, anti-diarrhea, anti-pyretic, anti-allergic, anti-hypertensive medications, analgesics, antibiotics, allopurinol, and others such as blood products and bone marrow growth factors. The Investigator may utilize erythropoietic factors, or blood or platelet transfusions at their discretion.

If the patient is thought to need >10 mg of prednisone or equivalent and their absolute neutrophil count is less than 500/μL, the Medical Monitor should be informed of the situation. If the patient is thought to need anticoagulation and their platelet count is <50,000/μL, the medical monitor should be informed of the situation.

### Rescue Medications, Treatments, and Procedures

Myeloid growth factors for patients with severe neutropenia and concurrent sepsis may be administered as per institutional guidelines.

### Screening Registration Procedures

The following assessments will be done prior to the treatment initiation:

Within 2 weeks prior to the treatment initiation, the following procedures will be completed:
Bone marrow aspirate/biopsy or cytologic evidence of relapsed or refractory AML (note: a biopsy can be used for confirmation of disease if taken within 4 weeks of randomization and patient should not be treated with any other therapy. If not, bone marrow aspirate/biopsy to be obtained within 2 weeks prior to treatment initiation)
Blood, buccal swabs (at screening/baseline only as a source of germline DNA), plasma and bone marrow aspirate/biopsy samples for biomarker evaluation (as applicable)
A complete medical history
ECOG PS (Oken M M, Creech R H, Tormey D, Horton J, Davis T E, McFadden E T et al. Toxicity and response criteria of the Eastern Cooperative Oncology Group. Am J Clin Oncol. 1982; 5:649-655) as close as possible to screening/baseline or within 48 hours before the first dose
   0=Fully active, able to carry on all pre-disease performance without restriction
   1=Restricted in physically strenuous activity but ambulatory and able to carry out work of a light or sedentary nature, e.g., light house work, office work
   2=Ambulatory and capable of all self-care but unable to carry out any work activities. Up and about more than 50% of waking hours
   3=Capable of only limited self-care, confined to bed or chair more than 50% of waking hours
   4=Completely disabled. Cannot carry on any self-care. Totally confined to bed or chair
   5=Dead
PRO by EORTC QLQ-C30
The following safety assessments/test will be performed:
   Baseline evaluation of symptoms and concomitant medications
   Physical exam (including height and weight) and vital signs (heart rate [HR], blood pressure [BP], body temperature, respiratory rate [RR])
   Clinical chemistry (including renal function), hematology and coagulation
   Assessment of LVEF (by TTE, MUGA or cardiac MRI)
   Baseline QTc determined by 12-lead ECG one replicate (Holter monitoring for Full ECG time points and ECG machines for Sparse ECG time points)
   Completion and submission of eligibility checklist

Within 1 week prior to treatment initiation:
Serum pregnancy test for women of child-bearing potential (test to be repeated on Day 1 (+/−1 day) at the beginning of every cycle)

### Randomization/Baseline Visit: Day 0

Patients will be randomized in 1:1 allocation ratio to HAM and CHAM Arms, respectively, in an open-label manner. During the randomization patients will be automatically assigned by IWRS to a corresponding treatment arm and stratification group. The randomization patient number will be provided by IWRS. The stratification factors are described above under Study Design.

The following baseline information will be collected from all randomized patients: AEs; Concomitant medications; Vital signs (weight, HR, BP, body temperature, RR); and PRO by EORTC QLQ-C30.

### Dosing and Administration

The amount of CPI-613® (devimistat) is based on the body surface area (BSA) of each patient. The BSA value will be calculated based on the Mostellar formula (BSA [m²]=square root of height [cm]×weight [kg]/3600) or other (preferred method is Mosteller formula). The weight assessment will be performed on Day 1 of each cycle. If the patient's body weight changes by >10% from baseline/screening, BSA should be recalculated and the dose adjusted for that given cycle.

CPI-613® (devimistat) drug product, solution is a sterile, nonpyrogenic, clear and colorless to light yellow solution suitable for IV administration. CPI-613® (devimistat) drug product is supplied in 10-mL USP type-I amber glass vial with 20 mm grey butyl stopper and royal blue flip off seal. Each mL contains: 50 mg of devimistat (CPI-613®) and 150 mg of triethanolamine (TEA). CPI-613® (devimistat) injection must be diluted from 50 mg/mL to 12.5 mg/mL with 5% dextrose (D-Glucose) in water (D5W) prior to administration (1 mL of CPI-613® (devimistat) diluted with 3 mL D5W). CPI-613® (devimistat) drug product is not compatible with saline solution.

The diluted drug product should be visually inspected for clarity. If haziness or precipitate is observed, do not use the diluted drug product for dosing. After dilution with sterile D5W, the solution should be clear and have a pH of 8.4 to 8.8. The diluted CPI-613® (devimistat) drug product has been found to be stable for 24 hours at room temperature (15 to 25° C.) with normal light exposure. The time of dose preparation and dose administration should be recorded to ensure that the drug is delivered within a 24-hour window.

Diluted CPI-613® (devimistat) drug product must be administered over 2 hours, concurrently with D5W at the rate of 125 mL/hour, by IV infusion via a central line catheter, using an infusion pump, that is free flowing and free of air in the dead space of the IV catheter. This is done to minimize vascular irritation, inflammation and acute toxicity of CPI-613® (devimistat) (Study NCL-049). Accidental co-administration of extra air in the dead space of IV catheters during administration of CPI-613® (devimistat) has demonstrated the potential to induce acute toxicity of CPI-613® (devimistat) according to animal studies (Study NCL-049). Also, accidental leakage of CPI-613® (devimistat) into the perivascular space during IV administration, which prolongs exposure of perivascular tissue to CPI-613® (devimistat), can induce significant local inflammation according to animal studies (Studies NCL-027 and NCL-030). To avoid local reactions at and around the site of administration, CPI-613® (devimistat) must be administered via a central IV catheter.

The following precautions must be taken when administering CPI-613® (devimistat):
1. Confirmation of the placement of the central IV line to ensure a lack of leakage of CPI-613® (devimistat)—into the perivascular space
2. Confirmation that the central IV line is free flowing
3. Confirmation that the central IV line is free of dead air space
4. Dilute CPI-613 (devimistat)—drug product with D5W, as instructed in the study-specific Pharmacy Manual
5. Administer CPI-613® (devimistat) by infusion over 2 hours, not as a bolus
6. After administration of CPI-613® (devimistat) flush the IV line with ˜10 mL of D5W to remove residual CPI-613® (devimistat).
Note: Flush volumes can be adjusted to accommodate the hold volumes of the infusion tubing for complete delivery of diluted drug product.

As CPI-613® (devimistat) can cause leaching of DEHP from IV infusion sets and IV bags, DEHP-containing IV infusion sets, IV bags or syringes should not be used for mixing or administration of CPI-613® (devimistat).

High dose cytarabine is administered by intravenous (IV) infusion at a dose of 1 g/m². Mitoxantrone is administered by IV infusion at a dose of 6 mg/m². After each dose of cytarabine and mitoxantrone, the central IV line must be flushed with D5W.

### Treatment/Intervention Plan

### Arm 1—CHAM: Study Group, Dose and Mode of Administration

### Induction Therapy (Up to 2 Cycles, Each Cycle: 2 Weeks)

First Cycle of Induction for CHAM (i.e. Arm 1) (and Full Induction Cycle 2 [subject to bone marrow results]):
a. Duration of each cycle is 14 days
b. CPI-613® (devimistat)* at 2,000 mg/m² is administered over 2 hours as a central line IV infusion QD (once per day) from Day 1 through Day 5 (total of 5 doses); Followed by:
c. High Dose Cytarabine* at 1 g/m², immediately following completion of CPI-613® (devimistat) infusion, is administered over 3 hours as a central line IV infusion (as per local approved prescription information/package insert instructions) Q12h starting on Day 3 through Day 5 (total of 5 doses). The time between completion of CPI-613® (devimistat) infusion and the start of Cytarabine infusion should be ≤30 minutes; Followed by:
d. Mitoxantrone* at 6 mg/m², following completion of High Dose Cytarabine infusion, is administered over 15 minutes as a central line IV infusion after the 1^{st}, 3^{rd} and 5^{th} doses of cytarabine starting on day 3 through day 5 (total of 3 doses). Mitoxantrone should be given as soon as possible, but no later than 30 minutes, after completion of each Cytarabine infusion
After each dose of CPI-613 (devimistat), the line must be flushed with D5W remove residual CPI-613® (devimistat). After each dose of Cytarabine and Mitoxantrone, the IV central line must be flushed with D5W as well.
Note: Flush volumes can be adjusted to accommodate the hold volumes of the infusion tubing for complete delivery of diluted drug product.
e. On Day 6 through Day 14, no therapy is administered.

| | Screening/ | | | | | | | | | |
|---|---|---|---|---|---|---|---|---|---|---|
| | Baseline | CHAM Induction Cycle 1 | | | | | | | | |
| Assessments | (Day-14-Day 0) | Day1 | Day2 | Day3 | Day4 | Day5 | Day6 | Day7 | Day 8 | Days 9-14 |
| Obtain ICF | √ | | | | | | | | | |
| Randomization | √ | | | | | | | | | |
| Medical History | √ | | | | | | | | | |
| Eligibility re-assessment for organ | | | | | | | | | | |
| function and performance status | | | | | | | | | | |

Study Drug Administration

| | | | | | |
|---|---|---|---|---|---|
| CPI-613 ® (devimistat) | √ | √ | √ | √ | √ |
| Cytarabine¹ | | | √ √ | √ √ | √ |
| Mitoxantrone² | | | √ | √ | √ |

Laboratory

| | | | | | | | | | |
|---|---|---|---|---|---|---|---|---|---|
| Serum pregnancy test for women of | √ | √ | | | | | | | |
| child-bearing potential | | | | | | | | | |
| Creatine and BUN^{3,4} | √ | | | | | | | | |
| 0 hr-24 hr (pre-dose) | | √ | √ | √ | √ | √ | | | |
| 24 hr ± 1 hr | | | | | | | √ | | |
| 48 hr ± 1 hr | | | | | | | | √ | |
| 72 hr ± 1 hr | | | | | | | | | √ |
| Clinical chemistry | √ | √ | √ | √ | √ | √ | √ | √ | √ |
| Hematology | √ | √ | √ | √ | √ | √ | √ | √ | √ |
| Coagulation⁵ | √ | √ | | | | | | | √ |

Efficacy

| | | |
|---|---|---|
| Study treatment efficacy (i.e. bone | Obtained within 4 | Obtained on Day 14⁷ of Induction Cycle 1 and upon count recovery |
| marrow aspirate/biopsy)⁶ | weeks prior to the | (defined as ANC > 1,000/μL, platelets > 100,000/μL) or on Day 42 if |
| | first dose | no count recovery is seen |

Patient Reported Outcome

| | | |
|---|---|---|
| EORTC QLQ-C30⁸ | √ | Obtained at baseline (prior to initiation of therapy) |

Pharmacokinetics

| | | | | | | | |
|---|---|---|---|---|---|---|---|
| Pharmacokinetics (PK)-Full PK⁹ | | | | | | | |
| 0 hr (pre CPI-613 ® (devimistat) | √ | √ | √ | √ | √ | | |
| 0.5 hr ± 15 mins | √ | | | | √ | | |
| 1 hr ± 15 mins | √ | | | | √ | | |
| 2 hr ± 15 mins | √ | √ | √ | √ | √ | | |
| 2.5 hr ± 15 mins | √ | | | | √ | | |
| 3 hr ± 30 mins | √ | | | | √ | | |
| 4 hr ± 30 mins | √ | | | | √ | | |
| 8 hr ± 1 hr | √ | | | | √ | | |
| 24 hr ± 1 hr | | | | | | √ | |
| 48 hr ± 1 hr | | | | | | | √ |
| Pharmacokinetics (PK)-Sparse PK | | | | | | | |
| 0 to 24 hrs | √ | | | | √ | | |

ECG

| | | | | | | | | |
|---|---|---|---|---|---|---|---|---|
| QTc measurement ECG | √ | | | | | | | |
| (one replicate) | | | | | | | | |
| 12 lead ECG-Full ECG | | | | | | | | |
| −30 mins-0 hr (pre-infusion) | | √ | √ | √ | √ | √ | | |
| 0.5 hr ± 15 mins | | √ | | | | √ | | |
| 1 hr ± 15 mins | | √ | | | | √ | | |
| 2 hr ± 15 mins | | √ | √ | √ | √ | √ | | |
| 2.5 hr ± 15 mins | | √ | √ | √ | √ | √ | | |
| 3 hr ± 30 mins | | √ | | | | √ | | |
| 4 hr ± 30 mins | | √ | | | | √ | | |
| 8 hr ± 1 hr | | √ | | | | √ | | |
| 24 hr ± 1 hr | | | | | | | √ | |
| 48 hr ± 1 hr | | | | | | | | √ |
| 12 lead ECG-Sparse ECG | | | | | | | | |
| 0 hr (pre-dose) | | √ | | | | √ | | |
| 2 hr ± 15 mins up to 24 hrs (post dose) | | √ | | | | √ | | |

Safety

| ECOG performance status¹⁰ | √ | | | | | | |
|---|---|---|---|---|---|---|---|
| Physical exam | √ √ | √ (only if clinically indicated) | | | | | only if |
| | (including height | | | | | | clinically |
| | and weight) | | | | | | indicated |
| Vital signs (heart rate, blood pressure, | √ | √ (immediately after study drug; re- | | | | | |
| body temperature, respiratory rate) | | examined only if clinically indicated) | | | | | |
| LVEF Assessment (TTE, MUGA, or | √ | | | | | | |
| MRI)¹¹ | | | | | | | |
| Adverse event review | √ | Continuous | | | | | |
| Serious adverse event review | √ | Continuous | | | | | |
| Evaluation of concomitant | √ | √ | √ | √ | √ | √ | √ |
| medications | | | | | | | |
| Evaluation of symptoms | √ | √ | √ | √ | √ | √ | √ |

Optional Assays (biomarkers)

| | | | | | | | |
|---|---|---|---|---|---|---|---|
| Buccal swabs¹² | √ | | | | | | |
| Plasma samples¹³ | | | | | | | |
| 0 hr (pre-dose) | | √ | √ | √ | √ | √ | |
| 2 hr ± 15 mins | | √ | √ | √ | √ | √ | |
| 4 hr ± 30 mins | | √ | √ | √ | √ | √ | |
| 6 hr ± 30 mins | | √ | √ | √ | √ | √ | |
| Bone marrow aspirate/biopsy¹⁶ | Obtained within 2 | | | | | | √ |
| | weeks prior to the | | | | | | |
| | first dose. | | | | | | |

¹Cytarabine administered Q12 h (total of 5 doses over Days 3-5). Time between completion of CPI-613 ® (devimistat) infusion and start of Cytarabine should be ≤30 minutes
²Mitoxantrone administered after 1st, 3rd, and 5th doses of Cytarabine as soon as possible, but no later than 30 minutes after completion of each Cytarabine infusion
³Creatinine Clearance (CrCl) will be calculated using the Cockroft Gault formula on Day 1 of each cycle.
⁴Creatinine and BUN to be checked within 24 hours prior to each dose administration and at 24, 48, and 72 hours post-last dose of CPI-613 ® (devimistat), Mitoxantrone, and Cytarabine (i.e. Days 6, 7, and 8). Only the results of creatinine are needed before CPI-613 ® (devimistat) is dosed
⁵Coagulation (PT/PTT/INR) to be completed Day 1 and weekly during Cycle 1 (Day 8)
⁶The response criteria will be assessed by standard criteria
⁷Bone marrow aspirate/biopsy on Day 14 of Induction Cycle 1 will only be collected if deemed appropriate by the investigator r and required for Induction Cycle 2 or Consolidation Cycle(s). A bone marrow aspirate/biopsy may be performed at any time if there is a concern of disease recurrence
⁸European Organization for Research and Treatment of Cancer Quality of Life Questionnaire C30
⁹North America Only
¹⁰ECOG: At least within 48 hours before patient randomization and on day 1 of Each cycle
¹¹TTE/MUGA/Cardiac MRI can be obtained within 2 weeks of consolidation as long as no other cardiac toxins have been administered
¹²Buccal Swabs are only collected at screening/baseline as a source of germline DNA
¹³Plasma Blood samples to be obtained just prior to administration of CPI-613 ® (devimistat) and at 2, 4, and 6 hours after the collection of the first sample on Days 1 to 5
¹⁴Bone marrow/aspirate biopsy samples to be obtained on Day 14 of Cycle 1 (samples may be collected ±1-2 days if collection falls on a weekend or holiday), upon count recovery or on Day 42

Second Cycle of Induction for CHAM (i.e. Arm 1) (based on bone marrow results):
Sites that do not perform day 14 bone marrow will not do induction cycle 2 (full or abbreviated) on any patients at the site.
A Full or Abbreviated Induction Cycle 2 may be performed based on the results of the bone marrow aspirate/biopsy performed on Day 14 after the start of Induction Cycle 1. If no Day 14 bone marrow aspirate/biopsy is obtained, no Induction Cycle 2 (Full or Abbreviated) will be given. Induction Cycle 2 (Full or Abbreviated) should be administered as soon as possible, but no later than 5 calendar days, following the Day 14 bone marrow/aspirate results. The decision for which Induction Cycle 2 (Full or Abbreviated) is performed will be based on the following criteria:
   Bone marrow aspirate/biopsy result shows <5% myeloblasts—no Induction Cycle 2 therapy will be given.
   Bone marrow aspirate/biopsy result shows ≥5% myeloblasts but <30% AND cellularity ≤20%—Abbreviated Induction Cycle 2 (3 days duration) will be given (see below).
If neither of the above criteria are met, a Full Induction Cycle 2 (repeat of Cycle 1 described above) therapy will be given.
In addition to the above guidance, patients from Arm 1 are only eligible to receive full Induction Cycle 2 therapy if they are hemodynamically stable i.e. mean arterial pressure (MAP) >60 mm Hg (not on pressors or fluid boluses), have maintained an ejection fraction (EF) sufficient to allow treatment with Mitoxantrone and are able to tolerate it in the opinion of the treating Investigator.
If no nadir marrow is obtained or after full Induction Cycle 2 therapy, a recovery bone marrow sample will be obtained when peripheral blood counts are consistent with remission (ANC >1,000/μL, platelets >100,000/μL and freedom from blood transfusions) or on Day 42 from the most recent chemotherapy, whichever comes first. If the results of this recovery marrow aspirate/biopsy show CR/CRi has been achieved, the patient can be given Consolidation Therapy (or if not eligible for Consolidation Therapy they can be given Maintenance Therapy). If no CR/CRi has been achieved, the patient is required to complete subsequent Safety Follow-up procedures and Long-Term Safety Follow-up procedures. If the recovery marrow is hypoplastic without evidence of persistent leukemia, an additional marrow can be done at the time of count recovery if discussed with the Medical Monitor.

| | CHAM Full Induction Cycle 2 | | | | | | | | |
|---|---|---|---|---|---|---|---|---|---|
| Assessments | Day1 | Day2 | Day3 | Day4 | Day5 | Day6 | Day7 | Day 8 | Days 9-14 |
| Eligibility re-assessment for organ | √ | | | | | | | | |
| function and performance status | | | | | | | | | |

Study Drug Administration

| | | | | | |
|---|---|---|---|---|---|
| CPI-613 ® (devimistat) | √ | √ | √ | √ | √ |
| Cytarabine¹ | | | √ √ | √ √ | √ |
| Mitoxantrone² | | | √ | √ | √ |

Laboratory

| | | | | | | | | |
|---|---|---|---|---|---|---|---|---|
| Serum pregnancy test for women of | √ | | | | | | | |
| child-bearing potential (±1 day) | | | | | | | | |
| Creatine and BUN^{3,4} | | | | | | | | |
| 0 hr-24 hr (pre-dose) | √ | √ | √ | √ | √ | | | |
| Clinical chemistry | √ | √ | √ | √ | √ | √ | √ | √ |
| Hematology | √ | √ | √ | √ | √ | √ | √ | √ |

Efficacy

| | |
|---|---|
| Study treatment efficacy (i.e. bone | Obtained upon count recovery |
| marrow aspirate/biopsy)⁵ | (defined as ANC > 1,000/μL, platelets > 100,000/μL) or |
| | on Day 42 if no count recovery is seen |

Patient Reported Outcome

| | |
|---|---|
| EORTC QLQ-C30⁶ | Obtained at the time of recovery bone marrow aspirate/biopsy after |
| | completion of induction cycle 1 upon count recovery (defined as |
| | ANC > 1,000/μL, platelets > 100,000/μL) or on Day 42 if no count |
| | recovery is seen |

Pharmacokinetics

| | | |
|---|---|---|
| Pharmacokinetics (PK)-Full PK⁷ | | |
| 0 hr (pre-dose) | √ | √ |
| 2 hr ± 15 mins | √ | √ |
| Pharmacokinetics (PK)-Sparse PK | | |
| 0 to 24 hrs | | √ |

ECG

| | | | | | |
|---|---|---|---|---|---|
| 12 lead ECG-Full ECG | | | | | |
| −30 mins-0 hr (pre-dose) | √ | | | | √ |
| 2 hr ± 15 mins | √ | √ | √ | √ | √ |
| 12 lead ECG-Sparse ECG | | | | | |
| 0 hr (pre-dose) | √ | | | | √ |
| 2 hr ± 15 mins (post dose) | √ | | | | √ |

Safety

| Physical exam | √ (only if clinically indicated) | | | | |
|---|---|---|---|---|---|
| Vital signs (heart rate, blood pressure, | √ (immediately after study drug; re- | | | | |
| body temperature, respiratory rate) | examined only if clinically indicated) | | | | |
| LVEF Assessment (TTE, MUGA, or | √ | | | | |
| MRI) | | | | | |
| Adverse event review | Continuous | | | | |
| Serious adverse event review | Continuous | | | | |
| Evaluation of concomitant | √ | √ | √ | √ | √ |
| medications | | | | | |
| Evaluation of symptoms | √ | √ | √ | √ | √ |

Optional Assays (biomarkers)

| | | | | | |
|---|---|---|---|---|---|
| Plasma samples⁸ | √ | √ | √ | √ | √ |
| 0 hr (pre-dose) | √ | √ | √ | √ | √ |
| 2 hr ± 15 mins | √ | √ | √ | √ | √ |
| 4 hr ± 30 mins | √ | √ | √ | √ | √ |
| 6 hr ± 30 mins | √ | √ | √ | √ | √ |

¹Cytarabine administered Q12 h (total of 5 doses over Days 3-5). Time between completion of CPI-613 ® (devimistat) infusion and start of Cytarabine should be ≤30 minutes
²Mitoxantrone administered after 1st, 3rd, and 5th doses of Cytarabine as soon as possible, but no later than 30 minutes after completion of each Cytarabine infusion
³Creatinine Clearance (CrCl) will be calculated using the Cockroft Gault formula on Day 1 of each cycle.
⁴Creatinine and BUN to be checked within 24 hours prior to each dose administration. Only the results of creatinine are needed before CPI-613 ® (devimistat) is dosed
⁵The response criteria will be assessed by standard criteria
⁶European Organization for Research and Treatment of Cancer Quality of Life Questionnaire C30
⁷North America Only
⁸Plasma Blood samples to be obtained just prior to administration of CPI-613 ® (devimistat) and at 2, 4, and 6 hours after the collection of the first sample on Days 1 to 5

### Abbreviated Induction Cycle 2—CHAM (Arm 1):

Duration of cycle is 14 days
CPI-613® (devimistat)* at 2000 mg/m² is administered over 2 hours as a central line IV infusion once daily on Day 1 through Day 3 (total of 3 doses) Followed by:
High Dose Cytarabine* at 1 g/m²′ immediately following completion of CPI-613® (devimistat) infusion, is administered over 3 hours as a central line IV infusion (as per institutional guidelines or package insert instructions) Q12h starting on Day 2 through Day 3 (total of 3 doses). The time between completion of CPI-613® (devimistat) infusion and the start of Cytarabine infusion should be <30 minutes Followed by:
Mitoxantrone* at 6 mg/m² following completion of High Dose Cytarabine, administered over 15 minutes as a central line IV infusion after the 1^{st} and 3^{rd} doses of Cytarabine (total of 2 doses). Mitoxantrone should be given as soon as possible, but no later than 30 minutes, after completion of each Cytarabine infusion.
* After each dose of CPI-613® (devimistat), the line must be flushed with D5W to remove residual CPI-613® (devimistat). After each dose of Cytarabine and Mitoxantrone, the IV central line must be flushed with D5W as well. Flush volumes can be adjusted to accommodate the hold volumes of the infusion tubing for complete delivery of diluted drug product.
On Day 4 through Day 14, no therapy is administered

| | CHAM Abbreviated Induction Cycle 2 | | | | | | | | |
|---|---|---|---|---|---|---|---|---|---|
| Assessments | Day1 | Day2 | Day3 | Day4 | Day5 | Day6 | Day7 | Day 8 | Days 9-14 |
| Eligibility re-assessment for organ | √ | | | | | | | | |
| function and performance status | | | | | | | | | |

Study Drug Administration

| | | | |
|---|---|---|---|
| CPI-613 ® (devimistat) | √ | √ | √ |
| Cytarabine¹ | | √ √ | √ |
| Mitoxantrone² | | √ | √ |

Laboratory

| | | | |
|---|---|---|---|
| Serum pregnancy test for women of | √ | | |
| child-bearing potential | | | |
| Creatine and BUN^{3,4} | | | |
| 0 hr-24 hr (pre-dose) | √ | √ | √ |
| Clinical chemistry | √ | √ | √ |
| Hematology | √ | √ | √ |

Efficacy

| | |
|---|---|
| Study treatment efficacy (i.e. bone | Obtained upon count recovery |
| marrow aspirate/biopsy)⁵ | (defined as ANC > 1,000/μL, platelets > 100,000/μL) or |
| | on Day 42 if no count recovery is seen |

Patient Reported Outcome

| | |
|---|---|
| EORTC QLQ-C30⁶ | Obtained at the time of recovery bone marrow aspirate/biopsy |

Pharmacokinetics

| | | |
|---|---|---|
| Pharmacokinetics (PK)-Full PK⁷ | | |
| 0 hr (pre-dose) | √ | √ |
| 2 hr ± 15 mins | √ | √ |
| Pharmacokinetics (PK)-Sparse PK | | |
| 0 to 24 hrs | | √ |

ECG

| | | |
|---|---|---|
| 12 lead ECG-Full ECG | | |
| −30 mins-0 hr (pre-dose) | √ | √ |
| 2 hr ± 15 mins | √ | √ |
| 12 lead ECG-Sparse ECG | | |
| 0 hr (pre-dose) | √ | √ |
| 2 hr ± 15 mins (post dose) | √ | √ |

Safety

| Physical exam | √ (only if clinically indicated) | | |
|---|---|---|---|
| Vital signs (heart rate, blood pressure, | √ (immediately after study drug; re- | | |
| body temperature, respiratory rate) | examined only if clinically indicated) | | |
| LVEF Assessment (TTE, MUGA, or | √ | | |
| MRI) | | | |
| Adverse event review | Continuous | | |
| Serious adverse event review | Continuous | | |
| Evaluation of concomitant | √ | √ | √ |
| medications | | | |
| Evaluation of symptoms | √ | √ | √ |

Optional Assays (biomarkers)

| | | | |
|---|---|---|---|
| Plasma samples⁸ | √ | √ | √ |
| 0 hr (pre-dose) | √ | √ | √ |
| 2 hr ± 15 mins | √ | √ | √ |
| 4 hr ± 30 mins | √ | √ | √ |
| 6 hr ± 30 mins | √ | √ | √ |

¹Cytarabine administered Q12 h (total of 3 doses over Days 2-3). Time between completion of CPI-613 ® (devimistat) infusion and start of Cytarabine should be ≤30 minutes
²Mitoxantrone administered after 1^{st} and 3rd doses of Cytarabine as soon as possible, but no later than 30 minutes after completion of each Cytarabine infusion
³Creatinine Clearance (CrCl) will be calculated using the Cockroft Gault formula on Day 1 of each cycle.
⁴Creatinine and BUN to be checked within 24 hours prior to each dose administrationof CPI-613 ® (devimistat). Only the results of creatinine are needed before CPI-613 ® (devimistat) is dosed
⁵The response criteria will be assessed by standard criteria
⁶European Organization for Research and Treatment of Cancer Quality of Life Questionnaire C30
⁷North America Only
⁸Plasma Blood samples to be obtained just prior to administration of CPI-613 ® (devimistat) and at 2, 4, and 6 hours after the collection of the first sample on Days 1 to 3

Consolidation Therapy (for Responders to Induction)—CHAM (i.e. Arm 1)

Patients who achieve CR or CRi according to standard response criteria for AML (Döhner H et al. 2017, supra), continue to meet the same organ function and performance status eligibility requirements (re-assessed prior to Day 1 of each cycle), and for whom allogeneic transplant is not possible or will be delayed by more than 6 weeks, will receive Consolidation treatment cycle(s). Therapy will start within 3 weeks of the establishment of CR or CRi and is identical to the Abbreviated Induction treatment cycles described above. Consolidation treatment will can be given in either the inpatient or outpatient setting as per institutional guidelines.

On completion of the first cycle of Consolidation, (i.e. complete count recovery or recovery to baseline counts and in the absence of disease progression or unacceptable toxicity) and if the patient continues to meet the same organ function and performance status eligibility requirements, a second Consolidation cycle will be given. No bone marrow aspirate/biopsy is expected to be taken between Consolidation cycles.

A summary of dosing schedule is provided in the following table.

### Dosing Schedule: CHAM (Arm 1)—Consolidation Treatment

| Day of Consolidation Therapy Cycle 1 | 1 | 2 | 3 | 4-14 |
|---|---|---|---|---|
| CPI-613 ® (devimistat) (2,000 mg/m²) | X | X | X | 0 |
| Cytarabine (1 g/m²) ^{a} | | X-X | X | 0 |
| Mitoxantrone (6 mg/m²) | | X | X | 0 |

^{a} Total of 3 doses (1 dose every 12 hours) over Days 2 and 3

All responders will receive hematopoietic stem cell transplantation (HSCT) whenever possible. Patients not eligible for HSCT and who were randomized to the CHAM arm are eligible to receive Maintenance Therapy.

| | CHAM Consolidation Cycle 1 & 2 | | | | | | | | |
|---|---|---|---|---|---|---|---|---|---|
| Assessments | Day1 | Day2 | Day3 | Day4 | Day5 | Day6 | Day7 | Day 8 | Days 9-14 |
| Eligibility re-assessment for organ | √ | | | | | | | | |
| function and performance status | | | | | | | | | |

Study Drug Administration

| | | | |
|---|---|---|---|
| CPI-613 ® (devimistat) | √ | √ | √ |
| Cytarabine¹ | | √ √ | √ |
| Mitoxantrone² | | √ | √ |

Laboratory

| | | | |
|---|---|---|---|
| Serum pregnancy test for women of | √ | | |
| child-bearing potential (±1 day) | | | |
| Creatine and BUN^{3,4} | | | |
| 0 hr-24 hr (pre-dose) | √ | √ | √ |
| Clinical chemistry | √ | √ | √ |
| Hematology | √ | √ | √ |

Efficacy

| | |
|---|---|
| Study treatment efficacy (i.e. bone | Obtained at the completion of all planned consolidation therapy or at |
| marrow aspirate/biopsy)⁵ | the time of disease recurrence |

Patient Reported Outcome

| | |
|---|---|
| EORTC QLQ-C30⁶ | Obtained at the completion of all consolidation therapy, every 3 |
| | months thereafter, and at the end of treatment (when possible) |

Pharmacokinetics

| | | |
|---|---|---|
| Pharmacokinetics (PK)-Full PK⁷ | | |
| 0 hr (pre-dose) | √ | √ |
| 2 hr ± 15 mins | √ | √ |
| Pharmacokinetics (PK)-Sparse PK | | |
| 0 to 24 hrs | √ | √ |
| | C1D1 | C2D3 |

ECG

| | | |
|---|---|---|
| 12 lead ECG-Full ECG | | |
| −30 mins-0 hr (pre-dose) | √ | √ |
| 2 hr ± 15 mins | √ | √ |
| 12 lead ECG-Sparse ECG | | |
| 0 hr (pre-dose) | √ | √ |
| 2 hr ± 15 mins (post dose) | √ | √ |

Safety

| ECOG performance staus | √ | | |
|---|---|---|---|
| Physical exam | √ (only if clinically indicated) | | |
| Vital signs (heart rate, blood pressure, | √ (immediately after study drug; re- | | |
| body temperature, respiratory rate) | examined only if clinically indicated) | | |
| LVEF Assessment (TTE, MUGA, or | √ | | |
| MRI) | | | |
| Adverse event review | Continuous | | |
| Serious adverse event review | Continuous | | |
| Evaluation of concomitant | √ | √ | √ |
| medications | | | |
| Evaluation of symptoms | √ | √ | √ |

Optional Assays (biomarkers)

| | | | |
|---|---|---|---|
| Plasma samples⁸ | | | |
| 0 hr (pre-dose) | √ | √ | √ |
| 2 hr ± 15 mins | √ | √ | √ |
| 4 hr ± 30 mins | √ | √ | √ |
| 6 hr ± 30 mins | √ | √ | √ |
| Bone marrow aspirate/biopsy⁹ | | | |

¹Cytarabine administered Q12 h (total of 3 doses over Days 2-3). Time between completion of CPI-613 ® (devimistat) infusion and start of Cytarabine should be ≤30 minutes
²Mitoxantrone administered after 1^{st} and 3rd doses of Cytarabine as soon as possible, but no later than 30 minutes after completion of each Cytarabine infusion
³Creatinine Clearance (CrCl) will be calculated using the Cockroft Gault formula on Day 1 of each cycle.
⁴Creatinine and BUN to be checked within 24 hours prior to each dose administrationof CPI-613 ® (devimistat). Only the results of creatinine are needed before CPI-613 ® (devimistat) is dosed
⁵The response criteria will be assessed by standard criteria
⁶European Organization for Research and Treatment of Cancer Quality of Life Questionnaire C30
⁷North America Only
⁸Plasma Blood samples to be obtained just prior to administration of CPI-613 ® (devimistat) and at 2, 4, and 6 hours after the collection of the first sample on Days 1 to 3
⁹Bone marrow/aspirate biopsy samples to be obtained at completion of all planned consolidation therapy cycles or at the time of disease progression

Maintenance Therapy (only for CHAM [Arm 1])

Patients randomized to CHAM (i.e. Arm 1) who obtain a CR or CRi according to standard response criteria for AML and who have completed all planned Induction and/or Consolidation Therapy (0, 1, or 2 cycles) but are not eligible for or refuse hematopoietic stem cell transplantation (HSCT) or for whom there will be a delay prior to HSCT of at least 6 weeks, will receive Maintenance Therapy. Maintenance Therapy is to commence within 3 weeks from count recovery and following completion of Induction/Consolidation Therapy. If the patient refuses Maintenance Therapy, they will be required to complete subsequent Safety Follow up procedures and Long-Term Safety Follow-up procedures.

CPI-613® (devimistat) drug product will be administered via a central line IV infusion at a dose of 2,500 mg/m² given on Day 1 through Day 5 of the cycle every 28 days. NO treatment will be administered on Day 6 through Day 28. This therapy is planned to be delivered in the outpatient setting.

NO High Dose Cytarabine or Mitoxantrone are given in Maintenance cycles. Maintenance is to be continued until disease recurrence, availability of stem cell transplant, the advent of intolerable side effects, or patient withdrawal of consent.

### Dosing Schedule: CHAM (Arm 1)—Maintenance Cycle

| Day of Maintenance Cycle | 1 | 2 | 3 | 4 | 5 | 6-28 |
|---|---|---|---|---|---|---|
| CPI-613 ® (devimistat) (2,500 mg/m²) | X | X | X | X | X | 0 |

| | CHAM Maintenance Cycle | | | | | | | | | | | | |
|---|---|---|---|---|---|---|---|---|---|---|---|---|---|
| Assessments | Day1 | Day2 | Day3 | Day4 | Day5 | Day6 | Day7 | Day 8 | Days 9-14 | EOT | SFU | L-T SFU | EOS |
| Eligibility re-assessment for organ | √ | | | | | | | | | | | | |
| function and performance status | | | | | | | | | | | | | |
| Survival follow-up | | | | | | | | | | √ | | √ | |

Study Drug Administration

| | | | | | |
|---|---|---|---|---|---|
| CPI-613 ® (devimistat) | √ | √ | √ | √ | √ |

Laboratory

| | | | | | | | | | |
|---|---|---|---|---|---|---|---|---|---|
| Serum pregnancy test for women of | √ | | | | | | | | |
| child-bearing potential (±1 day) | | | | | | | | | |
| Creatine and BUN^{1,2} | | | | | | | | | |
| 0 hr-24 hr (pre-dose) | √ | √ | √ | √ | √ | | | | √ |
| Clinical chemistry | √ | √ | √ | √ | √ | √ | √ | √ | √ |
| Hematology | √ | √ | √ | √ | √ | √ | √ | √ | |

Efficacy

| | |
|---|---|
| Study treatment efficacy (i.e. bone | Obtained upon completion of every 4^{th} cycle for the first year and then as clinically |
| marrow aspirate/biopsy)³ | indicated and at the time of progression |

Patient Reported Outcome

| | | | |
|---|---|---|---|
| EORTC QLQ-C30⁴ | √ | √ | √ |

Pharmacokinetics

| | | |
|---|---|---|
| Pharmacokinetics (PK)-Full PK⁵ | | |
| 0 hr (pre-dose) | √ | √ |
| 2 hr ± 15 mins | √ | √ |
| Pharmacokinetics (PK)-Sparse PK | | |
| 0 to 24 hrs | √ | √ |
| | M1D1 | M2D5 |

ECG

| | | |
|---|---|---|
| 12 lead ECG-Full ECG | | |
| −30 mins-0 hr (pre-dose) | √ | √ |
| 2 hr ± 15 mins | √ | √ |
| 12 lead ECG-Sparse ECG | | |
| 0 hr (pre-dose) | √ | √ |
| 2 hr ± 15 mins (post dose) | √ | √ |

Safety

| ECOG performance status⁶ | √ | | | | | | |
|---|---|---|---|---|---|---|---|
| Physical exam | √ (only if clinically indicated) | | | | | | |
| Vital signs (heart rate, blood pressure, | √ (immediately after study drug; re- | | | | | | |
| body temperature, respiratory rate) | examined only if clinically indicated) | | | | | | |
| Adverse event review | Continuous | | | | | | |
| Serious adverse event review | Continuous | | | | | | |
| Evaluation of concomitant | √ | √ | √ | √ | √ | √ | √ |
| medications | | | | | | | |
| Evaluation of symptoms | √ | √ | √ | √ | √ | √ | √ |

¹Creatinine Clearance (CrCl) will be calculated using the Cockroft Gault formula on Day 1 of each cycle.
²Crealinine and BUN to be checked within 24 hours prior to each dose administration of CPI-613® (devimistat). Only the results of creatinine are needed before CPI-613® (devimislat) is dosed
³The response criteria will be assessed by standard criteria
⁴European Organization for Research and Treatment of Cancer Quality of Life Questionnaire C30
⁵North America Only
⁶ECOG: At least within 48 hours before patient randomization and on day 1 of each cycle

### ARM 2—HAM: Control Group, Dose and Mode of Administration

### Induction Therapy (Up to 2 Cycles, Each Cycle: 2 Weeks)

Note: NO CPI-613® (devimistat) drug product will be administered to HAM (Arm 2) patients.

### Induction Cycle 1 (and Full Induction Cycle 2 [Subject to Bone Marrow Results]):

Duration of each cycle is 14 days
High Dose Cytarabine* at 1 g/m² is administered over 3 hours as a central line IV infusion (as per local approved information/package insert instructions) Q12h starting on Day 1 through Day 3 (total of 5 doses) Followed by:
Mitoxantrone* at 6 mg/m² is administered over 15 minutes as a central line IV infusion after the 1^{st}, 3^{rd} and 5^{th} doses of Cytarabine starting on Day 1 through Day 3 (total of 3 doses). Mitoxantrone should be given as soon as possible, but no later than 30 minutes, after completion of each Cytarabine administration. On Day 4 through Day 14, no therapy is administered
* After each dose of Cytarabine and Mitoxantrone, the IV central line must be flushed with D5W as well.
Note: Flush volumes can be adjusted to accommodate the hold volumes of the infusion tubing for complete delivery of diluted drug product.

### Induction Cycle 2 (Based on Bone Marrow Results):

A Full or Abbreviated Induction Cycle 2 may be performed based on the results of the bone marrow aspirate/biopsy performed on Day 14 after the start of Induction Cycle 1. If no Day 14 bone marrow aspirate/biopsy is obtained, no Induction Cycle 2 (Full or Abbreviated) will be given.

Induction Cycle 2 (Full or Abbreviated) should be administered as soon as possible, but no later than 5 calendar days, following the Day 14 bone marrow/aspirate results. The decision for which Induction Cycle 2 (Full or Abbreviated) is performed will be based on the following criteria:
Bone marrow aspirate/biopsy result shows <5% myeloblasts—no Induction Cycle 2 therapy will be given.
Bone marrow aspirate/biopsy result shows ≥5% myeloblasts but <30% AND cellularity ≤20%—Abbreviated Induction Cycle 2 (3 days duration) will be given (see below).

If neither of the above criteria are met, a Full Induction Cycle 2 (repeat of Cycle 1 described above) therapy will be given.
In addition to the above guidance, patients from Arm 2 are only eligible to receive Induction Cycle 2 therapy if they are hemodynamically stable i.e. MAP >60 mm Hg (not on pressors or fluid boluses), have maintained an EF sufficient to allow treatment with Mitoxantrone and are able to tolerate it in the opinion of the treating Investigator.
If no nadir marrow is obtained or after Induction Cycle 2 therapy, a recovery bone marrow sample will be obtained when peripheral blood counts are consistent with remission (ANC >1,000/μL, platelets >100,000/μL and freedom from blood transfusions) or on Day 42 from the most recent chemotherapy, whichever comes first.

If the results of this recovery marrow aspirate/biopsy show CR/CRi has been achieved, the patient can be given Consolidation Therapy; if no CR/CRi has been achieved, the patient is required to complete subsequent Safety Follow-up procedures and Long-Term Safety Follow-up procedures. If the recovery marrow is hypoplastic without evidence of persistent leukemia an additional marrow can be done at the time of count recovery if discussed with the Medical Monitor.

### Abbreviated Induction Cycle 2 (Duration of Cycle—14 Days):

High Dose Cytarabine* at 1 g/m² is administered over 3 hours as a central line IV infusion (as per institutional guidelines or package insert instructions) every 12 hours starting on Day 1 through Day 2 (total of 3 doses).
Followed by:
Mitoxantrone* at 6 mg/m² administered over 15 minutes as a central line IV infusion after the 1^{st} and 3^{rd} doses of Cytarabine (total of 2 doses). Mitoxantrone should be given as soon as possible, but no later than 30 minutes, after completion of each Cytarabine administration.
* After each dose of Cytarabine and Mitoxantrone, the IV central line must be flushed with D5W as well.
Note: Flush volumes can be adjusted to accommodate the hold volumes of the infusion tubing for complete delivery of diluted drug product.

A summary of the dosing schedules is provided in the following tables.

### Dosing Schedule: HAM (Arm 2)—Induction Cycle 1 and Full Induction Cycle 2 (Subject to Bone Marrow Results)

| Day of Induction | | | | |
|---|---|---|---|---|
| Cycle 1 or Full Cycle 2^{a} | 1 | 2 | 3 | 4-14 |
| Cytarabine (1 g/m²) ^{b} | X-X | X-X | X | 0 |
| Mitoxantrone (6 mg/m²) | X | X | X | 0 |

^{a}Induction Cycle 2 will not be given if Day 14 bone marrow aspirate/biopsy is not obtained
^{b} Total of 5 doses (1 dose every 12 hours) over Days 1 to 3

| | Screening/ | | | | | | | | | |
|---|---|---|---|---|---|---|---|---|---|---|
| | Baseline | HAM Induction Cycle 1 | | | | | | | | |
| Assessments | (Day-14-Day 0) | Day1 | Day2 | Day3 | Day4 | Day5 | Day6 | Day7 | Day 8 | Days 9-14 |
| Obtain ICF | √ | | | | | | | | | |
| Randomization | √ | | | | | | | | | |
| Medical History | √ | | | | | | | | | |
| Eligibility re-assessment for organ | | | | | | | | | | |
| function and performance status | | | | | | | | | | |

Study Drug Administration

| | | | |
|---|---|---|---|
| Cytarabine¹ | √ √ | √ √ | √ |
| Mitoxantrone² | √ | √ | √ |

Laboratory

| | | | | | |
|---|---|---|---|---|---|
| Serum pregnancy test for women of | √ | √ | | | |
| child-bearing potential | | | | | |
| Creatine and BUN^{3,4} | √ | | | | |
| 0 hr-24 hr (pre-dose) | | √ | √ | √ | |
| 24 hr ± 1 hr | | √ | | | |
| 48 hr ± 1 hr | | | √ | | |
| 72 hr ± 1 hr | | | | √ | |
| Clinical chemistry | √ | √ | √ | √ | |
| Hematology | √ | √ | √ | √ | |
| Coagulation⁵ | √ | √ | | | √ |

Efficacy

| | | |
|---|---|---|
| Study treatment efficacy (i.e. bone | Obtained within 4 | Obtained on Day 14⁷ of Induction Cycle 1 and upon count recovery |
| marrow aspirate/biopsy)⁶ | weeks prior to the | (defined as ANC > 1,000/μL, platelets > 100,000/μL) or on Day 42 if |
| | first dose | no count recovery is seen |

Patient Reported Outcome

| | | |
|---|---|---|
| EORTC QLQ-C30⁸ | √ | Obtained at baseline (prior to initiation of therapy) |
| | | and at the of recovery bone marrow biopsy and aspirate |

ECG

| | | | |
|---|---|---|---|
| QTc measurement ECG (one | √ | | |
| replicate) | | | |
| 12 lead ECG-Sparse ECG | | | |
| 0 hr (pre-dose) | | √ | √ |
| 2 hr ± 15 mins up to 24 hrs (post dose) | | √ | √ |

Safety

| ECOG performance status⁹ | √ | | | | |
|---|---|---|---|---|---|
| Physical exam | √ | √ (only if clinically indicated) | | | only if |
| | (including height | | | | clinically |
| | and weight) | | | | indicated |
| Vital signs (heart rate, blood pressure, | √ | √ (immediately after study drug; | | | |
| body temperature, respiratory rate) | | re-examined only if clinically | | | |
| | | indicated) | | | |
| LVEF Assessment (TTE, MUGA, or | √ | | | | |
| MRI)¹⁰ | | | | | |
| Adverse event review | √ | Continuous | | | |
| Serious adverse event review | √ | Continuous | | | |
| Evaluation of concomitant | √ | √ | √ | √ | √ |
| medications | | | | | |
| Evaluation of symptoms | √ | √ | √ | √ | √ |

Optional Assays (biomarkers)

| | | | | | |
|---|---|---|---|---|---|
| Buccal swabs¹¹ | √ | | | | |
| Plasma samples¹² | | | | | |
| 0 hr (pre-dose) | | √ | √ | √ | |
| 2 hr ± 15 mins | | √ | √ | √ | |
| 4 hr ± 30 mins | | √ | √ | √ | |
| 6 hr ± 30 mins | | √ | √ | √ | |
| Bone marrow aspirate/biopsy¹³ | Obtained within 2 | | | | √ |
| | weeks prior to the | | | | |
| | first dose. | | | | |

¹Cytarabine administered Q12 h (total of 5 doses over Days 1-3).
²Mitoxantrone administered after 1st, 3rd. and 5 th doses of Cytarabine as soon as possible, but no later than 30 minutes after completion of each Cytarabine infusion
³Creatinine Clearance (CrCl) will be calculated using the Cockroft Gault formula on Day 1 of each cycle.
⁴Creatinine and BUN to be checked within 24 hours prior to each dose administration and at 24, 48, and 72 hours post-dose of Mitoxantrone and Cytarabine (i.e. Days 1, 2, and 3). Only the results of creatinine are needed before dose administration
⁵Coagulation (PT/PTT/INR) to be completed Day 1 and weekly during Cycle 1 (Day 8)
⁶The response criteria will be assessed by standard criteria
⁷Bone marrow aspirate/biopsy on Day 14 of Induction Cycle 1 will only be collected if deemed appropriate by the investigator and required for Induction Cycle 2 or Consolidation Cycle(s). A bone marrow aspirate/biopsy may be performed at any time if there is a concern of disease recurrence
⁸European Organization for Research and Treatment of Cancer Quality of Life Questionnaire C30
⁹ECOG: At least within 48 hours before patient randomization and on day 1 of Each cycle
¹⁰TTE/MUGA/Cardiac MRI can be obtained within 2 weeks of consolidation as long as no other cardiac toxins have been administered
¹¹Buccal Swabs are only collected at screening/baseline as a source of germline DNA
¹²Plasma Blood samples to be obtained just prior to administration of cytarabine and at 2, 4, and 6 hours after the collection of the first sample on Days 1 to 3
¹³Bone marrow/aspirate biopsy samples to be obtained on Day 14 of Cycle 1 (samples may be collected ± 1-2 days if collection falls on a weekend or holiday), upon count recovery or on Day 42

| | HAM Full Induction Cycle 2 | | | | | | | | |
|---|---|---|---|---|---|---|---|---|---|
| Assessments | Day1 | Day2 | Day3 | Day4 | Day5 | Day6 | Day7 | Day 8 | Days 9-14 |
| Eligibility re-assessment for organ | √ | | | | | | | | |
| function and performance status | | | | | | | | | |

Study Drug Administration

| | | | |
|---|---|---|---|
| Cytarabine¹ | √ √ | √ √ | √ |
| Mitoxantrone² | √ | √ | √ |

Laboratory

| | | | |
|---|---|---|---|
| Serum pregnancy test for women of | √ | | |
| child-bearing potential (±1 day) | | | |
| Creatine and BUN^{3,4} | | | |
| 0 hr-24 hr (pre-dose) | √ | √ | √ |
| Clinical chemistry | √ | √ | √ |
| Hematology | √ | √ | √ |

Efficacy

| | |
|---|---|
| Study treatment efficacy (i.e. bone | Obtained upon count recovery |
| marrow aspirate/biopsy)⁵ | (defined as ANC > 1,000/μL, platelets > 100,000/μL) or |
| | on Day 42 if no count recovery is seen |

Patient Reported Outcome

| | |
|---|---|
| EORTC QLQ-C30⁶ | Obtained at baseline (prior to initiation of therapy) and at time of |
| | recovery bone marrow biopsy and aspirate |

ECG

| | | |
|---|---|---|
| 12 lead ECG-Sparse ECG | | |
| 0 hr (pre-dose) | √ | √ |
| 2 hr ± 15 mins (post dose) | √ | √ |

Safety

| Physical exam | √ (only if clinically | |
|---|---|---|
| | indicated) | |
| Vital signs (heart rate, blood pressure, | √ (immediately after | |
| body temperature, respiratory rate) | study drug; re- | |
| | examined only if | |
| | clinically indicated) | |
| LVEF Assessment (TTE, MUGA, or | √ | |
| MRI) | | |
| Adverse event review | Continuous | |
| Serious adverse event review | Continuous | |
| Evaluation of concomitant | √ | √ |
| medications | | |
| Evaluation of symptoms | √ | √ |

Optional Assays (biomarkers)

| | | | |
|---|---|---|---|
| Plasma samples⁷ | | | |
| 0 hr (pre-dose) | √ | √ | √ |
| 2 hr ± 15 mins | √ | √ | √ |
| 4 hr ± 30 mins | √ | √ | √ |
| 6 hr ± 30 mins | √ | √ | √ |

¹Cytarabine administered Q12 h (total of 5 doses over Days 1-3).
²Mitoxantrone administered after 1st, 3rd, and 5th doses of Cytarabine as soon as possible, but no later than 30 minutes after completion of each Cytarabine infusion
³Creatinine Clearance (CrCl) will be calculated using the Cockroft Gault formula on Day 1 of each cycle.
⁴Creatinine and BUN to be checked within 24 hours prior to each dose administration and at 24, 48, and 72 hours post-dose of mitoxantrone and cytarabine (i.e., Days 1, 2, and 3). Only the results of creatinine are needed before dose administration
⁵The response criteria will be assessed by standard criteria
⁶European Organization for Research and Treatment of Cancer Quality of Life Questionnaire C30
⁷Plasma Blood samples to be obtained just prior to administration of cytarabine and at 2, 4, and 6 hours after the collection of the first sample on Days 1 to 3

### Dosing Schedule: HAM (Arm 2)—Abbreviated Induction Cycle 2 (Subject to Bone Marrow Results)

| Day of Abbreviated Induction Cycle 1^{a} | 1 | 2 | 3-14 |
|---|---|---|---|
| Cytarabine (1 g/m²) ^{b} | X-X | X | 0 |
| Mitoxantrone (6 mg/m²) | X | X | 0 |

^{a}Induction Cycle 2 will not be given if Day 14 bone marrow aspirate/biopsy is not obtained.
^{b} Total of 3 doses (1 dose every 12 hours) over Days 1 and 2

| | HAM Abbreviated Induction Cycle 2 | | | | | | | | |
|---|---|---|---|---|---|---|---|---|---|
| Assessments | Day1 | Day2 | Day3 | Day4 | Day5 | Day6 | Day7 | Day 8 | Days 9-14 |
| Eligibility re-assessment for organ | √ | | | | | | | | |
| function and performance status | | | | | | | | | |

Study Drug Administration

| | | |
|---|---|---|
| Cytarabine¹ | √ √ | √ √ |
| Mitoxantrone² | √ | √ |

Laboratory

| | | |
|---|---|---|
| Serum pregnancy test for women of | √ | |
| child-bearing potential (±1 day) | | |
| Creatine and BUN^{3,4} | | |
| 0 hr-24 hr (pre-dose) | √ | √ |
| Clinical chemistry | √ | √ |
| Hematology | √ | √ |

Efficacy

| | |
|---|---|
| Study treatment efficacy (i.e. bone | Obtained upon count recovery |
| marrow aspirate/biopsy)⁵ | (defined as ANC > 1,000/μL, platelets > 100,000/μL) or |
| | on Day 42 if no count recovery is seen |

Patient Reported Outcome

| | |
|---|---|
| EORTC QLQ-C30⁶ | Obtained at baseline (prior to initiation of therapy) and at time of |
| | recovery bone marrow biopsy and aspirate |

ECG

| | | |
|---|---|---|
| 12 lead ECG-Sparse ECG | | |
| 0 hr (pre-dose) | √ | √ |
| 2 hr ± 15 mins (post dose) | √ | √ |

Safety

| Physical exam | √ (only if | |
|---|---|---|
| | clinically | |
| | indicated) | |
| Vital signs (heart rate, blood pressure, | √ (immediately | |
| body temperature, respiratory rate) | after | |
| | study drug; re- | |
| | examined only | |
| | if clinically | |
| | indicated) | |
| LVEF Assessment (TTE, MUGA, or | √ | |
| MRI) | | |
| Adverse event review | Continuous | |
| Serious adverse event review | Continuous | |
| Evaluation of concomitant | √ | √ |
| medications | | |
| Evaluation of symptoms | √ | √ |

Optional Assays (biomarkers)

| | | | |
|---|---|---|---|
| Plasma samples⁷ | | | |
| 0 hr (pre-dose) | √ | √ | √ |
| 2 hr ± 15 mins | √ | √ | √ |
| 4 hr ± 30 mins | √ | √ | √ |
| 6 hr ± 30 mins | √ | √ | √ |

¹Cytarabine administered Q12 h (total of 5 doses over Days 1-3).
²Mitoxantrone administered after 1^{st}, 3rd, and 5th doses of Cytarabine as soon as possible, but no later than 30 minutes after completion of each Cytarabine infusion
³Creatinine Clearance (CrCl) will be calculated using the Cockroft Gault formula on Day 1 of each cycle.
⁴Creatinine and BUN to be checked within 24 hours prior to first cytarabine dose administration. Only the results of creatinine are needed before dose administration
⁵The response criteria will be assessed by standard criteria
⁶European Organization for Research and Treatment of Cancer Quality of Life Questionnaire C30
⁷Plasma Blood samples to be obtained just prior to administration of cytarabine and at 2, 4, and 6 hours after the collection of the first sample on Days 1 to 2

### Consolidation Therapy (for Responders to Induction)—HAM (Arm 2)

Patients who achieve CR or CRi according to standard response criteria for AML (Döhner H et al. 2017, supra), continue to meet the same organ function and performance status eligibility requirements (re-assessed prior to Day 1 of each cycle), and for whom allogeneic transplant is not possible or will be delayed by more than 6 weeks, will receive Consolidation treatment cycle(s). Therapy will start within 3 weeks of the establishment of CR or CRi and is identical to the Abbreviated Induction treatment cycles described above. Consolidation treatment will can be given in either the inpatient or outpatient setting as per institutional guidelines.

On completion of the first cycle of Consolidation, (i.e. complete count recovery or recovery to baseline counts and in the absence of disease progression or unacceptable toxicity) and if the patient continues to meet the same organ function and performance status eligibility requirements, a second Consolidation cycle will be given. No bone marrow aspirate/biopsy is expected to be taken between Consolidation cycles.

A summary of dosing schedules is provided in the following table.

### Dosing Schedule: HAM (Arm 2)—Consolidation Treatment

| Day of Consolidation Therapy Cycle 1 | 1 | 2 | 3-14 |
|---|---|---|---|
| Cytarabine (1 g/m²) ^{a} | X-X | X | 0 |
| Mitoxantrone (6 mg/m²) | X | X | 0 |

^{a} Total of 3 doses (1 dose every 12 hours) over Days 1 and 2

| | HAM Consolidation Cycles 1 and 2 | | | | | | | | | | | | |
|---|---|---|---|---|---|---|---|---|---|---|---|---|---|
| | | | | | | | | | Days | | | LT-S and | |
| Assessments | Day1 | Day2 | Day3 | Day4 | Day5 | Day6 | Day7 | Day 8 | 9-14 | EOT | SFU | QOL FU | EOS |
| Eligibility re-assessment for organ | √ | | | | | | | | | | | | |
| function and performance status | | | | | | | | | | | | | |
| Survival follow-up | | | | | | | | | | √ | | √ | |

Study Drug Administration

| | | |
|---|---|---|
| Cytarabine¹ | √ √ | √ |
| Mitoxantrone² | √ | √ |

Laboratory

| | | | |
|---|---|---|---|
| Serum pregnancy test for women of | √ | | |
| child-bearing potential (±1 day) | | | |
| Creatine and BUN^{3,4} | | | |
| 0 hr-24 hr (pre-dose) | √ | √ | |
| Clinical chemistry | √ | √ | √ |
| Hematology | √ | √ | √ |

Efficacy

| | |
|---|---|
| Study treatment efficacy (i.e. bone | Obtained at the completion of all planned consolidation therapy |
| marrow aspirate/biopsy)⁵ | or at the time of disease recurrence |

Patient Reported Outcome

| | | | |
|---|---|---|---|
| EORTC QLQ-C30⁶ | √ | √ | √ |

ECG

| | | |
|---|---|---|
| 12 lead ECG-Sparse ECG | | |
| 0 hr (pre-dose) | √ | √ |
| 2 hr ± 15 (post dose) | √ | √ |

Safety

| | |
|---|---|
| ECOG performance status⁷ | √ |

| | |
|---|---|
| Physical exam | √ (only if |
| | clinically |
| | indicated) |
| Vital signs (heart rate, blood pressure, | √ (immediately |
| body temperature, respiratory rate) | after study |
| | drug; re- |
| | examined only |
| | if clinically |
| | indicated) |

| | |
|---|---|
| LVEF Assessment (TTE, MUGA, or | √ |

| MRI) | | | |
|---|---|---|---|
| Adverse event review | Continuous | | |
| Serious adverse event review | Continuous | | |
| Evaluation of concomitant | √ | √ | √ |
| medications | | | |
| Evaluation of symptoms | √ | √ | √ |

Optional Assays (biomarkers)

| | | |
|---|---|---|
| Plasma samples⁹ | | |
| 0 hr (pre-dose) | √ | √ |
| 2 hr ± 15 mins | √ | √ |
| 4 hr ± 30 mins | √ | √ |
| 6 hr ± 30 mins | √ | √ |
| Bone marrow aspirate/biopsy¹⁰ | | |

¹Cytarabine administered Q12 h (total of 3 doses over Days 1-2).
²Mitoxantrone administered after 1^{st} and 3rd doses of Cytarabine as soon as possible, but no later than 30 minutes after completion of each Cytarabine infusion
³Creatinine Clearance (CrCl) will be calculated using the Cockroft Gault formula on Day 1 of each cycle.
⁴Creatinine and BUN to be checked within 24 hours prior to dose administration. Only the results of creatinine are needed before dose administration
⁵The response criteria will be assessed by standard criteria
⁶European Organization for Research and Treatment of Cancer Quality of Life Questionnaire C30
⁷ECOG: At least within 48 hours before patient randomization and on day 1 of each cycle
⁸TTE/MUGA/Cardiac MRI can be obtained within 2 weeks of consolidation as long as no other cardiac toxins have been administered. Prior to consolidation cycle 2 TTE/MUGA/cardiac MRI is at the discretion of the investigator
⁹Plasma Blood samples to be obtained just prior to administration of cytarabine and at 2, 4, and 6 hours after the collection of the first sample on Days 1 to 3
¹⁰Bone marrow/aspirate biopsy samples to be obtained at the completion of all planned consolidaton therapy cycles or at the time of disease recurrence

Standard of Care Study Procedures (Arm 2)
Baseline bone marrow aspirate/biopsy obtained within 2 weeks prior to treatment initiation.
Nadir bone marrow aspirate/biopsy obtained on Day 14 (+/−3 days) of Induction Cycle 1.
Recovery bone marrow aspirate/biopsy obtained following Induction therapy upon count recovery (ANC >1,000/μL, platelet count >100,000/μL and freedom from blood transfusions) or on Day 42 from the last Induction cycle if count recovery is not seen.
Bone marrow aspirate/biopsy at time of completion of all planned consolidation therapy.
Bone marrow aspirate/biopsy every 4^{th} cycle during maintenance therapy for the 1^{st} year (i.e. after cycle 4, 8 and 12, Arm 1 only).
Bone marrow aspirate/biopsy at any time if there is concern for progression of disease should be performed as standard of care at the treating sites.
Assessment of LVEF by TTE or MUGA or cardiac MRI, sufficient to safely administer mitoxantrone. Subjects must have a LVEF ≥45%.
Assessment of Screening/Baseline QTc (by 12-lead ECG)—measured via a single 12-lead ECG not requiring central reading. QTc interval to be ≤450 msec and ≤470 msec for males and females, respectively.
Physical exams, vital signs, ECOG PS, evaluation of symptoms and medications are assessed as defined in the schedule of events for each cycle.

### Bone Marrow Samples (Bone Marrow Aspirate/Biopsy)

Up to 10 unstained bone marrow biopsy slides will be collected (minimum of 5 slides from aspirate and a minimum of 2 slides from biopsy) and aspirate collected in K2EDTA tube from those obtained at Screening/Baseline, Day 14, Day 42 or any of those taken every 3 to 4 months during disease surveillance.

These slides will be used for IHC staining for PDKs, PDH, KGDH, SOD2 and CD79a. They will also be used as a source of material for RNA and whole exome sequencing should the bone marrow aspirate material be inadequate.

### Plasma Samples

Peripheral blood samples will be collected at Screening or Baseline and at 2, 4, and 6 hours after the collection of the first sample on Days 1 to 5 in CHAM and Days 1 to 3 in HAM.

### Pharmacokinetic Analysis Blood Sampling (CHAM [Arm 1] Only)

Upon randomization through the IWRS system, patients from CHAM (Arm 1) will participate in the PK analyses, as described below. Blood samples will be collected for PK analysis of CPI-613® (devimistat) and its metabolites CPI-2850 and CPI-1810. PK samples can be drawn from the central IV line (or peripheral line if unable to draw from the central IV line) after flushing with 10 ml of D5W.

Two groups of patients from CHAM (Arm 1) will participate in the PK analyses (PK sample draws will be taken immediately after ECG recordings):

Group A—Full PK Analysis (CHAM [Arm 1] North American Region Only)

Randomized patients will be assigned to the Full PK analysis until 24 evaluable patients are obtained. Consequently, more than 24 patients may potentially be assigned to the Full PK analysis to obtain 24 evaluable patients. A patient will be considered evaluable for PK analysis if a PK blood sample has been collected at one or more time points post CPI-613® (devimistat) drug product. Once 24 evaluable patients have been obtained, randomized patients will be assigned to Sparse PK analysis (see below).

Site participation in the Full PK analyses will be dependent on hospital site, experience and the site's ability to collect a more detailed blood profile over a 7-day (168 hours) window.

The total number of patients participating in the Full PK analysis will not exceed approximately 25 patients. The goal of Full PK analysis will not only be to assess Cₘₐₓ, Cₘᵢₙ, AUC₀₋₁, AUC_{inf}, T_{1/2}, Tₘₐₓ, CL and V_{d} for both CPI-613® (devimistat) and its metabolites CPI-2850 and CPI-1810, but also assess PK for clinical efficacy and safety. In addition, components of the Cytarabine and Mitoxantrone standard of care regimen may also be evaluated.

### Full PK Blood Sampling Schedule (CHAM [Arm 1]—North American Region Only)

| Phase, | | | |
|---|---|---|---|
| Cycle, Day | Timepoints | N | Comment |
| Induction, | 0 hr (pre-dose) | 24 patients | Pre-CPI-613 ® |
| Cycle 1, | | enrolled and | (devimistat) infusion |
| Day 1 | 0.5 hr ± 10 min | randomized to | During CPI-613 ® |
| | | CHAM arm | (devimistat) infusion |
| | 1 hr ± 15 min | (Arm 1). Must | During CPI-613 ® |
| | | have agreed to | (devimistat) infusion |
| | 2 hr ± 15 min | participate in | During CPI-613 ® |
| | | FULL PK | (devimistat) infusion |
| | 2.5 hr ± 15 min | analysis | End of CPI-613 ® |
| | | | (devimistat) infusion |
| | 3 hr ± 30 min | | Post CPI-613 ® |
| | | | (devimistat) infusion |
| | 4 hr ± 30 min | | Post CPI-613 ® |
| | | | (devimistat) infusion |
| | 8 hr ± 1 hr | | Post CPI-613 ® |
| | | | (devimistat) infusion |
| Induction, | 0 hr (pre-dose) | 24 patients | Pre-CPI-613 ® |
| Cycle 1, | | above | (devimistat) infusion |
| Day 2 | 2 hr ± 15 min | | End of CPI-613 ® |
| | | | (devimistat) infusion |
| Induction, | 0 hr (pre-dose) | 24 patients | Pre-CPI-613 ® |
| Cycle 1, | | above | (devimistat) infusion |
| Day 3 | 2 hr ± 15 min | | End of CPI-613 ® |
| | | | (devimistat) infusion |
| Induction, | 0 hr (pre-dose) | 24 patients | Pre-CPI-613 ® |
| Cycle 1, | | above | (devimistat) infusion |
| Day 4 | 2 hr ± 15 min | | End of CPI-613 ® |
| | | | (devimistat) infusion |
| Induction, | 0 hr (pre-dose) | 24 patients | Pre-infusion |
| Cycle 1, | 0.5 hr ± 10 min | above | During CPI-613 ® |
| Day 5 | | | (devimistat) infusion |
| | 1 hr ± 15 min | | During CPI-613 ® |
| | | | (devimistat) infusion |
| | 2 hr ± 15 min | | End of CPI-613 ® |
| | | | (devimistat) infusion |
| | 2.5 hr ± 15 min | | Post CPI-613 ® |
| | | | (devimistat) infusion |
| | 3 hr ± 30 min | | Post CPI-613 ® |
| | | | (devimistat) infusion |
| | 4 hr ± 30 min | | Post CPI-613 ® |
| | | | (devimistat) infusion |
| | 8 hr ± 1 hr | | Post CPI-613 ® |
| | | | (devimistat) infusion |
| Induction, | 24 hr ± 1 hr | 24 patients | Day 6 |
| Cycle 1, | | above | |
| Day 6 | | | |
| Induction, | 48 hr ± 1 hr | 24 patients | Day 7 |
| Cycle 1, | | above | |
| Day 7 | | | |
| Induction, | 0 hr (pre-dose) | 24 patients | Pre-CPI-613 ® |
| Cycle 2, | | above | (devimistat) infusion |
| Day 1 | 2 hr ± 15 min | | End of CPI-613 ® |
| | | | (devimistat) infusion |
| Induction, | 0 hr (pre-dose) | 24 patients | Pre-CPI-613 ® |
| Cycle 2, | | above | (devimistat) infusion |
| Day 5 | 2 hr ± 15 min | | End of CPI-613 ® |
| (or Day 3)^{a} | | | (devimistat) infusion |
| Consolidation, | 0 hr (pre-dose) | 24 patients | Pre-CPI-613 ® |
| Cycle 1, Day 1 | | above | (devimistat) infusion |
| | 2 hr ± 15 min | | End of CPI-613 ® |
| | | | (devimistat) infusion |
| Consolidation, | 0 hr (pre-dose) | 24 patients | Pre-CPI-613 ® |
| Cycle 1, Day 3 | | above | (devimistat) infusion |
| | 2 hr ± 15 min | | End of CPI-613 ® |
| | | | (devimistat) infusion |
| Consolidation, | 0 hr (pre-dose) | 24 patients | Pre-CPI-613 ® |
| Cycle 2, Day 1 | | above | (devimistat) infusion |
| | 2 hr ± 15 min | | End of CPI-613 ® |
| | | | (devimistat)-infusion |
| Consolidation, | 0 hr (pre-dose) | 24 patients | Pre-CPI-613 ® |
| Cycle 2, Day 3 | | above | (devimistat) infusion |
| | 2 hr ± 15 min | | End of CPI-613 ® |
| | | | (devimistat) infusion |
| Maintenance, | 0 hr (pre-dose) | 24 patients | Pre-CPI-613 ® |
| Cycle 1, Day 1 | | above | (devimistat) infusion |
| | 2 hr ± 15 min | | End of CPI-613 ® |
| | | | (devimistat) infusion |
| Maintenance, | 0 hr (pre-dose) | 24 patients | Pre-CPI-613 ® |
| Cycle 1, Day 5 | | above | (devimistat) infusion |
| | 2 hr ± 15 min | | End of CPI-613 ® |
| | | | (devimistat)-infusion |
| Maintenance, | 0 hr (pre-dose) | 24 patients | Pre-CPI-613 ® |
| Cycle 2, Day 1 | | above | (devimistat) infusion |
| | 2 hr ± 15 min | | End of CPI-613 ® |
| | | | (devimistat) infusion |
| Maintenance, | 0 hr (pre-dose) | 24 patients | Pre-CPI-613 ® |
| Cycle 2, Day 5 | | above | (devimistat) infusion |
| | 2 hr ± 15 min | | End of CPI-613 ® |
| | | | (devimistat)-infusion |

^{a}Day 5 if 5 days of treatment or Day 3 if 3 days of treatment in Induction Cycle 2 (i.e. Abbreviated Induction cycle)

Group B—Sparse PK Analysis and Blood Sampling—CHAM (Arm 1) Excluding Full PK Analysis Patients.

Upon randomization through the IWRS system, the first 25 patients from CHAM (Arm 1) will be assigned to Sparse PK Analysis (Group B). Subsequent patients from the NA region will be assigned to Group A (Full PK analysis, described above) until 24 evaluable patients have been obtained. Thereafter, the remaining randomized patients (not including the NA region) will participate in the Sparse PK Analysis. A patient will be considered evaluable for PK analysis if a PK blood sample has been collected at one or more time points post CPI-613® (devimistat) drug product.

A PK sample for Sparse PK analysis will be collected from each patient at 6 possible time points identified in the table below. Each sample will be collected at each time point within 0 to 24 hours with time counted from the end of IV infusion of CPI-613® (devimistat) drug product on the specified day. In addition to measurement of CPI-613® (devimistat) and metabolites CPI-2850 and CPI-1810, the components of Cytarabine and Mitoxantrone as standard of care regimen may also be evaluated.

### Sparse PK Blood Sampling Schedule—CHAM (Arm 1) Excluding Full PK Analysis Patients.

| Sample | Phase, Cycle, Day | Time Point Window |
|---|---|---|
| 1 | Induction Cycle 1 | 0-24 hr |
| | Day 1 | |
| 2 | Induction Cycle 2 | 0-24 hr |
| | Day 5 (or Day 3)^{a} | |
| 3 | Consolidation Cycle 1 | 0-24 hr |
| | Day 1 | |
| 4 | Consolidation Cycle 2 | 0-24 hr |
| | Day 3 | |
| 5 | Maintenance Cycle 1 | 0-24 hr |
| | Day 1 | |
| 6 | Maintenance Cycle 2 | 0-24 hr |
| | Day 5 | |

^{a}Day 5 if 5 days of treatment or Day 3 if 3 days of treatment in Induction Cycle 2 (i.e. Abbreviated Induction cycle)

### ECG Analysis

All patients will have an ECG measurement taken at Screening. Two groups of patients (both treatment arms) will undergo ECG analyses (ECG recordings will be taken immediately prior to PK sample draws).

Group A—Full ECG Analysis (CHAM [Arm 1]—North American Region Only). The same 24 patients randomized to CHAM (Arm 1) who are assigned to the Full PK analysis (Group A) will also undergo a Full ECG analysis using 12-lead Holter monitoring. The start and stop times of each recording will be documented.
Twelve-lead ECGs will be recorded at defined intervals from approximately 30 minutes prior to dosing of CPI-613® (devimistat) (up to 30 minutes prior to 0 hour) on Cycle 1, Day 1 to 24 hours after dosing on Day 2. Patients will be placed in a supine position for at least 10 minutes prior to time points for ECG recording (and PK sampling). ECG recordings will be taken immediately prior to the PK sample draw at each time point. ECG intervals for the full ECG analysis will be measured at a central ECG laboratory that is fully blinded to time points and patient identification. The 12-lead ECGs in these patients will be recorded using a Global Instrumentation (Manlius, N.Y., USA) M12R ECG continuous 12-lead digital recorder.
For Cycle 1, Day 1 (8 time points) and Cycle 1, Day 5 (up to 10 time points, including 48 hours), continuous 12-lead digital ECG data will be recorded and stored onto SD memory cards. At each protocol-specified time point, QT and RR intervals will be measured from three independent 12-lead ECG recordings (i.e. 3 replicates) these and ECGs will be uploaded onto a central eRT server. Safety ECGs can be printed on-site with the same device. At each protocol-specified time point, QT and RR intervals will be measured by ERT for a maximum of 10 replicate ECGs. The QTc interval will be derived using Fridericia's formula from the preceding RR interval and the QT interval in each beat, and the median QTcF in each replicate will be calculated. The schedule for Full ECG analysis is provided in the table below.

### Full ECG Analysis Schedule (CHAM [Arm 1] Only—North American Region Only)

| Phase, | | | |
|---|---|---|---|
| Cycle, Day | Time Points | N | Comment |
| Induction, | 0 hr (up to 30 min | 24 patients | Pre-CPI-613 ® |
| Cycle 1, | pre-dose) | (i.e. the same | (devimistat) infusion |
| Day 1 | 0.5 hr ± 15 min | 24 patients | During CPI-613 ® |
| | | enrolled and | (devimistat) infusion |
| | 1 hr ± 15 min | randomized to | During CPI-613 ® |
| | | CHAM | (devimistat) infusion |
| | 2 hr ± 15 min | arm (Arm 1) | End of CPI-613 ® |
| | | for Full PK | (devimistat) infusion |
| | 2.5 hr ± 15 min | analysis) | Post CPI-613 ® |
| | | | (devimistat) infusion |
| | 3 hr ± 30 min | | Post CPI-613 ® |
| | | | (devimistat) infusion |
| | 4 hr ± 30 min | | Post CPI-613 ® |
| | | | (devimistat) infusion |
| | 8 hr ± 30 min | | Post CPI-613 ® |
| | | | (devimistat) infusion |
| Induction, | 0 hr (pre-dose) | 24 patients | Pre-CPI-613 ® |
| Cycle 1, | 2 hr ± 15 min | above | (devimistat) infusion |
| Day 2 | | | End of CPI-613 ® |
| | | | (devimistat) infusion |
| Induction, | 0 hr (pre-dose) | 24 patients | Pre-CPI-613 ® |
| Cycle 1, | 2 hr ± 15 min | above | (devimistat) infusion |
| Day 3 | | | End of CPI-613 ® |
| | | | (devimistat) infusion |
| Induction, | 0 hr (pre-dose) | 24 patients | Pre-CPI-613 ® |
| Cycle 1, | 2 hr ± 15 min | above | (devimistat) infusion |
| Day 4 | | | End of CPI-613 ® |
| | | | (devimistat) infusion |
| Induction, | 0 hr (pre-dose) | 24 patients | Pre-infusion |
| Cycle 1, | 0.5 hr ± 15 min | above | During CPI-613 ® |
| Day 5 | | | (devimistat) infusion |
| | 1 hr ± 15 min | | During CPI-613 ® |
| | | | (devimistat) infusion |
| | 2 hr ± 15 min | | End of CPI-613 ® |
| | | | (devimistat) infusion |
| | 2.5 hr ± 15 min | | Post CPI-613 ® |
| | | | (devimistat) infusion |
| | 3 hr ± 30 min | | Post CPI-613 ® |
| | | | (devimistat) infusion |
| | 4 hr ± 30 min | | Post CPI-613 ® |
| | | | (devimistat) infusion |
| | 8 hr ± 30 min | | Post CPI-613 ® |
| | | | (devimistat) infusion |
| Induction, | 24 hr ± 1 hr | 24 patients | Day 6 |
| Cycle 1, Day 6 | | above | |
| Induction, | 48 hr ± 1 hr | 24 patients | Day 7 |
| Cycle 1, Day 7 | | above | |
| Induction, | 0 hr (pre-dose) | 24 patients | Pre-CPI-613 ® |
| Cycle 2, Day 1 | 2 hr ± 15 min | above | (devimistat) infusion |
| | | | End of devimistat |
| | | | (CPI-613 ®) infusion |
| Induction, | 0 hr (pre-dose) | 24 patients | Pre-CPI-613 ® |
| Cycle 2, | 2 hr ± 15 min | above | (devimistat) infusion |
| Day 5 (or | | | End of CPI-613 ® |
| Day 3)^{a} | | | (devimistat) infusion |
| Consolidation, | 0 hr (pre-dose) | 24 patients | Pre-CPI-613 ® |
| Cycle 1, | 2 hr ± 15 min | above | (devimistat) infusion |
| Day 1 | | | End of CPI-613 ® |
| | | | (devimistat) infusion |
| Consolidation, | 0 hr (pre-dose) | 24 patients | Pre-CPI-613 ® |
| Cycle 1, | 2 hr ± 15 min | above | (devimistat) infusion |
| Day 3 | | | End of CPI-613 ® |
| | | | (devimistat)infusion |
| Consolidation, | 0 hr (pre-dose) | 24 patients | Pre-CPI-613 ® |
| Cycle 2, | 2 hr ± 15 min | above | (devimistat) infusion |
| Day 1 | | | End of CPI-613 ® |
| | | | (devimistat) infusion |
| Consolidation, | 0 hr (pre-dose) | 24 patients | Pre-CPI-613 ® |
| Cycle 2, | 2 hr ± 15 min | above | (devimistat) infusion |
| Day 3 | | | End of CPI-613 ® |
| | | | (devimistat) infusion |
| Maintenance, | 0 hr (pre-dose) | 24 patients | Pre-CPI-613 ® |
| Cycle 1, | 2 hr ± 15 min | above | (devimistat) infusion |
| Day 1 | | | End of CPI-613 ® |
| | | | (devimistat) infusion |
| Maintenance, | 0 hr. (pre-dose) | 24 patients | Pre-CPI-613 ® |
| Cycle 1, | 2 hr ± 15 min | above | (devimistat) infusion |
| Day 5 | | | End of CPI-613 ® |
| | | | (devimistat) infusion |
| Maintenance, | 0 hr (pre-dose) | 24 patients | Pre-CPI-613 ® |
| Cycle 2, | 2 hr ± 15 min | above | (devimistat) infusion |
| Day 1 | | | End of CPI-613 ® |
| | | | (devimistat) infusion |
| Maintenance, | 0 hr (pre-dose) | 24 patients | Pre-CPI-613 ® |
| Cycle 2, | 2 hr ± 15 min | above | (devimistat) infusion |
| Day 5 | | | End of CPI-613 ® |
| | | | (devimistat) infusion |

^{a}Day 5 if 5 days treatment or Day 3 if 3 days treatment in Induction Cycle 2 (i.e. Abbreviated Induction cycle)

Group B—Sparse ECG Analysis—All CHAM (Arm 1) Patients Except for North American Region (Full ECG Analysis Patients) and All HAM (Arm 2) Patients. Upon randomization through the IWRS system, the first 25 patients from CHAM (Arm 1; not from NA region) will be assigned to Sparse ECG Analysis (Group B). Subsequent patients (from the NA region) will be assigned to Group A (Full ECG analysis, described above) until 24 evaluable patients have been obtained. Thereafter, the remaining randomized patients (not including the NA region) will participate in the Sparse ECG Analysis.
Twelve-lead ECGs will be recorded at defined intervals. Patients will be placed in a supine position for at least 10 minutes prior to time points for ECG recording (and PK sampling). Sparse ECG measurement will be measured by a central ECG laboratory.
At each protocol-specified time point, QT and RR intervals will be measured from each of the three independent 12-lead ECG recordings (i.e. 3 replicates). The QTc interval will be derived using Fridericia's formula from the preceding RR interval and the QT interval in each beat and the median QTcF in each replicate will be calculated. The mean across medians from all replicates will be used as the patient's reportable value at that time point.
All medications administered to patients from 4 hours prior to the first ECG reading through all time points will be recorded (including doses and time of administration). Measurement of PR and QRS intervals will be performed semi-automatically on 3 sequential beats from each of the 3 replicates. The mean value will be calculated for each replicate and then the mean of these used as the patient's reportable value at the timepoint (adapted from Darpo et. al.). The schedule for Sparse ECG analysis is provided in the table below.
   Sparse ECG Measurement Schedule—All CHAM (Arm 1) (excluding Full ECG Measurement Patients) and All HAM (Arm 2) Patients).

| Phase, Cycle, Day | Time points | N | Comment |
|---|---|---|---|
| Induction, Cycle 1, | 0 hr | Approximately 476 | Pre-infusion for Arm 1 and 2 |
| Day 1 | 2 hr ± 15 min | patients = First 25 | (CHAM and HAM) and post- |
| | | enrolled patients + up | infusion at 2 hours for Arm 1 |
| | | to 451 patients | and 2 (CHAM and HAM) |
| | | enrolled after | |
| | | assignment of patients | |
| | | for Full ECG analysis) | |
| Induction, Cycle 1, | 0 hr | Approximately 476 | Pre-infusion for Arm 1 and 2 |
| Day 5 (or Day 3^{a}) | 2 hr ± 15 min | patients | (CHAM and HAM) and post- |
| | | | infusion at 2 hours for Arm 1 |
| | | | and 2 (CHAM and HAM) |
| Induction, Cycle 2, | 0 hr | Approximately 476 | Pre-infusion for Arm 1 and 2 |
| Day 1 | 2 hr ± 15 min | patients | (CHAM and HAM) and post- |
| | | | infusion at 2 hours for Arm 1 |
| | | | and 2 (CHAM and HAM) |
| Induction, Cycle 2, | 0 hr | Approximately 476 | Pre-infusion for Arm 1 and 2 |
| Day 5 (or Day 2^{a} or | 2 hr ± 15 min | patients | (CHAM and HAM) and post- |
| Day 3^{b}) | | | infusion at 2 hours for Arm 1 |
| | | | and 2 (CHAM and HAM) |
| Consolidation, Cycle 1, | 0 hr | Approximately 476 | Pre-infusion for Arm 1 and 2 |
| Day 1 | 2 hr ± 15 min | patients | (CHAM and HAM) and post- |
| | | | infusion at 2 hours for Arm 1 |
| | | | and 2 (CHAM and HAM) |
| Consolidation, Cycle 1, | 0 hr | Approximately 476 | Pre-infusion for Arm 1 and 2 |
| Day 3 (or Day 2^{a}) | 2 hr ± 15 min | patients | (CHAM and HAM) and post- |
| | | | infusion at 2 hours for Arm 1 |
| | | | and 2 (CHAM and HAM) |
| Consolidation, Cycle 2, | 0 hr | Approximately 476 | Pre-infusion for Arm 1 and 2 |
| Day 1 | 2 hr ± 15 min | patients | (CHAM and HAM) and post- |
| | | | infusion at 2 hours for Arm 1 |
| | | | and 2 (CHAM and HAM) |
| Consolidation, Cycle 2, | 0 hr | Approximately 476 | Pre-infusion for Arm 1 and 2 |
| Day 3 (or Day 2^{a}) | 2 hr ± 15 min | patients | (CHAM and HAM) and post- |
| | | | infusion at 2 hours for Arm 1 |
| | | | and 2 (CHAM and HAM) |
| Maintenance^{c}, Cycle 1, | 0 hr | Approximately 476 | Pre-infusion for Arm 1 and 2 |
| Day 1 | 2 hr ± 15 min | patients | (CHAM and HAM) and post- |
| | | | infusion at 2 hours for Arm 1 |
| | | | and 2 (CHAM and HAM) |
| Maintenance^{c}, Cycle 1, | 0 hr | Approximately 476 | Pre-infusion for Arm 1 and 2 |
| Day 5 | 2 hr ± 15 min | patients | (CHAM and HAM) and post- |
| | | | infusion at 2 hours for Arm 1 |
| | | | and 2 (CHAM and HAM) |
| Maintenance^{c}, Cycle 2, | 0 hr | Approximately 476 | Pre-infusion for Arm 1 and 2 |
| Day 1 | 2 hr ± 15 min | patients | (CHAM and HAM) and post- |
| | | | infusion at 2 hours for Arm 1 |
| | | | and 2 (CHAM and HAM) |
| Maintenance^{c}, Cycle 2, | 0 hr | Approximately 476 | Pre-infusion for Arm 1 and 2 |
| Day 5 | 2 hr ± 15 min | patients | (CHAM and HAM) and post- |
| | | | infusion at 2 hours for Arm 1 |
| | | | and 2 (CHAM and HAM) |

^{a}For patients on ARM 2-HAM
^{b}For patients on ARM 1-CHAM Day 5 if 5 days of treatment or Day 3 if 3 days of treatment in Induction Cycle 2 (i.e. Abbreviated Induction cycle)
^{c}ARM 1-CHAM only

### Cardiac Marker (Troponin I)

24 patients in each arm (Arm A and Arm B) will be evaluated for elevated Troponin I. The schedule is detailed in the tables below.

### Cardiac Marker Schedule

### ARM A

| Phase, Cycle, Day | Time Points | N | Comment |
|---|---|---|---|
| Induction, Cycle 1, | 0 hr (up to 30 min | 24 patients | Pre-CPI-613 ® |
| Day 1 | pre-dose) | | (devimistat) infusion |
| Induction, Cycle 1, | Last dose of study | 24 patients | Post-Mitoxantrone |
| Day 5 | drug (+30 min) | | infusion |

### ARM B

| Phase, Cycle, Day | Time Points | N | Comment |
|---|---|---|---|
| Induction, Cycle 1, | 0 hr (up to 30 min | 24 patients | Pre-Cytarabine infusion |
| Day 1 | pre-dose) | | |
| Induction, Cycle 1, | Last dose of study | 24 patients | Post-Mitoxantrone |
| Day 3 | drug (+30 min) | | infusion |

### Administration of Questionnaires/Other Instruments for Patient-Reported Outcomes (PRO)

PRO by EORTC QLQ-C30 will be administered at the following time points:
Screening/Baseline
Time of recovery marrow
Upon completion of Consolidation therapy (if applicable)
Every 3 months after completion of consolidation (if applicable) as follows:
   For CHAM Arm: During the maintenance phase, Long-Term Safety and QOL Follow-up period and End of Treatment Visit and/or End of Study Visit
   For HAM Arm: During the Long-Term Safety and QOL Follow-up period, End of Treatment Visit and/or End of Study Visit

### Clinical Laboratory Evaluations

All safety evaluations will be conducted by local laboratories and databased.

Clinical Chemistry

Clinical chemistry assessment includes the following parameters: Glucose, Creatinine, Total Protein, Albumin, Sodium, Potassium, Chloride⁻, Magnesium, Calcium, Phosphorus, CO₂, Uric Acid, Blood Urea Nitrogen (BUN), AST/SGOT, ALT/SGPT, Alkaline Phosphatase (ALP), Lactate Dehydrogenase, Total Bilirubin. Renal function will be assessed by CrCl calculated using the CockCroft-Gault formula).

Hematology

Complete blood count with differential.

Coagulation

Prothrombin time (PT), partial thromboplastin time (PTT) and INR.

### Buccal Swabs

Buccal swabs will be collected at Screening or Baseline as a source of germline DNA for comparison with tumor-obtained leukemic cells in bone marrow aspirate/biopsy at diagnosis for possible mutations. Please refer to the Laboratory Manual for collection and handling procedures. Buccal swabs will also be bio-banked for analyses and comparison.

### End of Treatment Visit

No assessments will be carried out during this visit. The following information will be collected for each patient, as appropriate: Last dosing date for each study drug administered; Patient status at end of treatment; Reason for discontinuing treatment, if applicable; HSCT type and date, if applicable; Non-compliance/protocol violations, if applicable; Whether the patient will continue to Survival Follow-up; Patient-Reported Outcomes (PRO) EORTC QLQ-C30.

### Safety Follow-Up

Upon permanent discontinuation from the study treatment for any reason, a Safety Follow-Up visit (to include review of AEs/SAEs, assess ECOG PS, symptoms and concomitant medication, and perform safety laboratory tests) will occur approximately 30 (±2) days after completion of the last dosing interval of CPI-613® (devimistat) and/or protocol-required therapies.

### Long-Term Safety and QOL Follow-Up Visit

Patients will be followed every month for the first 3 months and every 3 months thereafter up to month 12 to collect survival status. These contacts may be performed over the phone. Every attempt should be done to collect survival status.

### End of Study Visit

Adverse events, as reported by the patient or observed by the Investigator, will be recorded and follow-up completed on AEs and/or SAEs. In addition, the following information will be collected for each patient, as appropriate: Study discontinuation date; Reason for permanently discontinuing the study, if applicable; Non-compliance/protocol violations, if applicable; For patients who withdrew consent, whether they also withdrew consent to have biological specimens stored for future research; Date last patient known to be alive; Death Details (if applicable, i.e., death date and primary cause of death (to include primary AE details, if applicable)); Patient-Reported Outcomes (PRO) EORTC QLQ-C30.

### Duration of Treatment

Patients randomized to the control arm (HAM, Arm 2) will continue to receive planned treatment until completion of all required Induction/Consolidation therapy cycles and then observed as part of the Long-Term Follow-Up, as determined by the Sponsor. Patients randomized to the study arm (CHAM, Arm 1) following completion of all planned Induction and/or Consolidation therapy cycles will continue to receive CPI-613® (devimistat) drug product during Maintenance Cycle(s) until disease recurrence, availability of stem cell transplant, the advent of intolerable side effects, or patient withdrawal of consent. The duration of participation for each patient is approximately 12 months.

Observations after the Intervention

Patients will be followed for at least 12 months following completion of all study treatments or until death from any cause, whichever is longer. A bone marrow biopsy will be obtained every 4 months for the first year following completion of all consolidation therapy.

### Toxicities/Side Effects

An adverse event (AE) is defined as an undesired medical occurrence in a patient receiving a study treatment and which does not necessarily have a causal relationship with this treatment. An AE can therefore be any unfavorable sign (including an abnormal laboratory finding), symptom, or disease temporarily associated with the study treatment, whether or not related to the investigational product.

An AE or suspected adverse reaction is considered “serious” if, in the view of either the investigator or sponsor, it results in any of the following outcomes: a) results in or leads to death (except from progression of AML disease), b) is life-threatening (meaning the patient was at immediate risk of death from the reaction as it occurred i.e. it does not include a reaction that hypothetically might have caused death had it occurred in a more severe form), c) admitted to the hospital overnight, excluding hospitalization for planned procedures, AML treatment and due to social indications, d) results in persistent or significant disability/incapacity or substantial disruption of the ability to conduct normal life functions, e) is a congenital anomaly/birth defect, or f) is otherwise considered a medically important event.

Important medical events that may not result in death, be life-threatening, or require hospitalization may be considered serious when, based upon appropriate medical judgment, they may jeopardize the patient and may require medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization, or the development of drug dependency or drug abuse.

Events not to be considered as adverse events or serious adverse events are a) medical conditions present at the initial trial visit that do not worsen in severity or frequency during the trial are defined as baseline medical conditions and NOT to be considered AEs, and b) death resulting from disease progression, hospitalization for the study-related treatment or PK sample, and planned hospitalization or hospitalization due to social indication, should not be reported as an SAE.

An AE is classified as Grade 1 (Mild) if it requires minimal or no treatment and does not interfere with the participant's daily activities. An AE is classified as Grade 2 (Moderate) if it a) results in a low level of inconvenience or concern with the therapeutic measures, or b) causes some interference with functioning. An AE is classified as Grade 3 (Severe) if it interrupts a participant's usual daily activity and may require systemic drug therapy or other treatment. An AE is classified as Grade 4 (life threatening or disabling) if the patient is at risk of death at the time of the event. An AE is classified as Grade 5 if death is related to the AE.

### Evaluation of an Adverse Event (AE)

Definitely Related—A clinical event follows a reasonable temporal sequence from the time of Investigational Product (CPI-613®) administration, cannot be reasonably explained by other factors such as the subject's medical condition, or concurrent medical condition, or concurrent medication(s) AND either occurs following active Investigational Product (CPI-613®) administration, improves on stopping the Investigational Product (CPI-613®), or reappears on re-exposure

Probably Related—A clinical event follows a reasonable temporal sequence from the time of Investigational Product (CPI-613®) administration. The toxicity cannot be reasonably explained by other factors such as the subjects clinical state or concurrent medical condition, or concurrent medication(s).

Possibly Related—A clinical event follows a reasonable temporal sequence from the time of active Investigational Product (CPI-613®) administration, but could also have been produced by other factors such as the subjects clinical state, or concurrent medical condition, or concurrent medication(s).

Unlikely Related—A clinical event is doubtfully related to active Investigational Product (CPI-613®). The event was most likely related to other factors such as the subject's clinical state, or concurrent medical condition, or concurrent medication(s) or the temporal relationship to the administration and/or exposure suggests that a causal relationship is unlikely.

Not Related—The event is clearly due to causes other than the Investigational Product (CPI-613®).

Adverse events that are probably related to devimistat include Nausea, Vomiting, Diarrhea, Increased creatinine, Neutropenia, Hyperkalemia, Hypoalbuminemia, Hypophosphatemia, Hyponatremia, Fatigue, Platelet count decreased, Renal failure (reversible), Glomerular filtration rate (decreased), Hemoglobin (anemia). Adverse events possibly related to devimistat include ALP (elevated or decreased ALP), Anorexia, ALT (SGPT) (elevated or decreased ALT), AST (SGOT) (elevated or decreased AST), Bilirubin (hyperbilirubinemia), Calcium (hypercalcemia, hypocalcemia), Flushing, Leukocytes (elevated or decreased leukocyte count), Lymphopenia, Neutrophils (neutropenia), Platelets (thrombocytopenia), Potassium (hypokalemia), Sodium (hypernatremia), Cardiac Arrhythmia (bradycardia).

Any medical condition that is present at the time that the patient is screened will be considered as baseline and not reported as an AE. However, if the study patient's condition deteriorates at any time during the study, it will be recorded as an AE. For the purposes of expedited safety reporting, ‘reasonable possibility’ means there is evidence to suggest a causal relationship between the drug and the AE. Suspected Unexpected Serious Adverse Reactions (SUSARs) will be recorded in the data collection system throughout the study.

### Dose Modifications for Cytarabine and Mitoxantrone

General considerations for dose modifications for Cytarabine and Mitoxantrone:
Dose modifications will be based on the toxicity requiring the largest dose reduction. Dose modifications may be acceptable after a discussion with the Medical Monitor.
Dose modifications may be made for individual drugs if, in the judgment of the Investigator, the toxicity is attributable to one drug.
All dose reductions must be recorded with the reason for the reduction.
Any modification should be based on labs obtained the day of drug administration and should follow guidelines below. If dose modifications are required and toxicities subsequently resolve, Cytarabine or Mitoxantrone may be re-introduced in full dose for the next cycle.
Other dose modifications may be acceptable after discussion with the Medical Monitor. No dose reductions will be made for myelosuppression.

Cytarabine dose modification:
Bilirubin >2×ULN: dose reduction by 50% (500 mg/m²)
CrCl (creatinine clearance) >2 mg/dL or <50 mL/min using the CockCroft-Gault formula: dose reduction by 50% (500 mg/m²).

Mitoxantrone dose modification:
Bilirubin >3×ULN: dose reduction by 25% (4.5 mg/m²)
Bilirubin >5×ULN: omit Mitoxantrone
ALT/SGPT/AST/SGOT >300 U/L: dose reduction by 50%.

### Dose Modifications for CPI-613® (Devimistat) (for Toxicities Attributed as at Least Possibly Related to CPI-613® (Devimistat) Dose Adjustment are as Follows):

| | |
|---|---|
| Toxicity | |
| and Intensity | |
| as per CTCAE^{a} | Supportive Care and Dose Adjustment Guidelines |

Nausea, Acute (common)

| | |
|---|---|
| Grade 1 or 2 | Maintain dose and schedule. Rule out other causes. |
| (If intolerable or | Utilize anti-nausea medications including 5-HT3 |
| persistent Grade 2 | antagonists. |
| not responsive to | |
| supportive care, | |
| follow guidelines | |
| for Grade 3) | |
| Grade 3-4 | Rule out other causes. Utilize anti-nausea |
| | medications including 5-HT3 antagonists. |
| | Interrupt CPI-613 dosing until resolved to Grade 1 |
| | or baseline and reduce CPI-613 dose by 50%. |

Diarrhea (common)

| | |
|---|---|
| Grade 1 | Maintain dose and schedule. Rule out other causes |
| | including drug effects. Treat per institutional |
| | guidelines with anti-diarrheals. |
| Grade 2 | Rule out other causes including drug effects. Treat |
| | per institutional guidelines with anti-diarrheals. |
| | Interrupt CPI-613 dosing until resolved to Grade |
| | 1 or baseline. |
| | For first occurrence, restart CPI-613 at current dose. |
| | For ≥second occurrence, reduce dose by 25%. |
| Grade 3 or 4 | Interrupt CPI-613 dosing until resolved to Grade 1 |
| | or baseline and patient is clinically stable. Reduce |
| | CPI-613 dose by 50%. |

Acute Kidney Injury

| | |
|---|---|
| Grade 1 | Maintain dose and schedule. |
| Grade 2 | Hold dose until resolved to grade 1 or baseline and |
| | resume CPI-613 at a 25% dose reduction. |
| Grade ≥ 3 | Hold dose until resolved to grade 1 or baseline and |
| | resume CPI-613 at a 50% dose reduction. |

Non-Hematological
Adverse Events (During Induction and Consolidation)

| | |
|---|---|
| Grade 1 | Maintain dose and schedule. |
| Grade 2 | Hold dose until resolved to grade 1 or baseline and |
| | resume CPI-613 at a 25% dose reduction. |
| Grade 3 or 4 | Hold dose until resolved to grade 1 or baseline and |
| | resume CPI-613 at a 50% dose reduction. |

Other Adverse Events
Including Hematological (During Maintenance Therapy)

| | |
|---|---|
| Grade 1 | Maintain dose and schedule of CPI-613. |
| Grade 2 | Hold dose until resolved to grade 1 or baseline and |
| | resume CPI-613 at a 25% dose reduction. |
| Grade 3 or 4 | Hold dose until resolved to grade 1 or baseline and |
| | resume CPI-613 at a 50% dose reduction. |

CTCAE: Common Terminology Criteria for Adverse Events
^{a}Version 5.0 or later (https://ctep.cancer.gov/protocolDevelopment/electronic_applications/ctc.htm)

### Statistics

### Description of Statistical Methods

One-sided tests will be used at a significance level equal to 0.025. Two-sided confidence intervals will be computed for a coverage of 0.95. Time to event outcomes (“survival times”) will be described by treatment arm using the Kaplan-Meier method. Subjects who have not had the event of interest at the time of the analysis will be censored at the time of the last follow up. Summary statistics will be provided by treatment arm in terms of the number of events, median and 95% confidence interval, and survival probabilities at specific time points (such as 1 year, 2 years, etc.). Survival curves will be plotted by treatment arm, and compared with a log-rank test stratified by performance status, age and refractory versus relapsed disease. A stratified Cox regression model will be used to estimate the hazard ratio and its 95% CI, as well as to adjust the comparison for baseline covariates. Binary outcomes will be described by proportions by treatment arm and compared with a Cochran-Mantel-Haenszel (CMH) test stratified by performance status, age and refractory versus relapsed disease A logistic regression model will be used to adjust the comparison for baseline covariates.

The primary endpoint is CR (Complete Remission). Attainment of a CR is associated with an improved quality of life and longer survival in AML patients, and is required for patients to be able to proceed with a Reduced-Intensity Conditioning allogenic stem cell transplant, the only curative treatment modality for relapsed or refractory AML in patients 50 years or older. The treatment arm will be compared with control arm in terms of CR. The primary analysis will be a re-randomization test (Simon R, “Restricted randomization designs in clinical trials,” Biometrics, 1979, 35, 503-12) based on the CMH test-statistic, stratified by performance status, age and refractory versus relapsed disease. The re-randomization approach fixes all data except the treatment labels at their observed values, regenerates the randomization sequence using the minimization algorithm (Buyse M, “Centralized Treatment Allocation in Comparative Clinical Trials,” Applied Clinical Trials, 2000, 9, 32-37), and computes the test statistic corresponding to those reshuffled assignments. This process is repeated a large number of times, and a p-value is calculated as the proportion of re-randomized trials whose test statistic is at least as extreme as the observed one from the original assignments.

The trial design includes two interim analyses, and one final analysis. Both interim analyses are only performed for the primary endpoint CR rate with the intent to stop the trial if the difference in CR between arms is not sufficiently promising; there is no intention to stop the study early if efficacy is shown. The futility boundaries are conceived as non-binding boundaries allowing the DMC to decide independently at the timing of the interim analysis, taking all available data into account, whether the study should continue or stop.

The first interim analysis will be performed when 167 patients are evaluable for response. This analysis will take place approximately 14 months after the first randomization. The only endpoint analyzed at this interim analysis will be CR. The significance level to be used for this interim analysis will be determined using an O'Brien-Fleming type Lan-DeMets boundary for efficacy, and a Pocock type Lan-DeMets boundary for futility. Assuming an information fraction of 33% for CR at this interim analysis, the significance level for efficacy will be 0.0001 and it will be reached if the difference in CR is larger than 26.7%. The significance level for futility is 0.32 and it will be reached if the difference in CR is smaller than 3.3%. If, based on this interim analysis, the CR difference is not sufficiently promising, consideration will be given to stopping the trial; otherwise, the trial will proceed. There is however no intention to stop the trial for efficacy with only 167 patients.

The second interim analysis will be performed when 333 patients are evaluable for response. This analysis will take place approximately 25 months after the first randomization. The only endpoint analyzed at this interim analysis will be CR. As with the 1^{st} interim analysis, the significance level to be used for this interim analysis will be determined using an O'Brien-Fleming type Lan-DeMets boundary for efficacy, and a Pocock type Lan-DeMets boundary for futility. Assuming an information fraction of 67% for CR at this interim analysis, the significance level for efficacy will be 0.006 and it will be reached if the difference in CR is larger than 12.8%. The significance level for futility is 0.094 and it will be reached if the difference in CR is smaller than 6.7%. If, based on this interim analysis, the CR difference is not sufficiently promising, consideration will be given to stopping the trial; otherwise, the trial will proceed.

The final analysis will be performed when 500 patients are evaluable for response. This analysis will take place approximately 36 months after the first randomization. At that time, the primary endpoint of CR will be analyzed first, with a significance level determined using an O'Brien-Fleming type Lan-DeMets boundary for efficacy equal to 0.023, which will be reached if the difference in CR is larger than 8.3%. In case efficacy is declared for CR, the Sponsor may consider to file for accelerated approval based on CR while patients are further followed to collect OS data.

The secondary endpoints are (a) OS (overall survival), defined as the duration from the date of randomization to the date of death from any cause, (b) CR+CRh (complete remission and complete remission with partial hematologic recovery), and (c) safety. OS and CR+CRh are the two key secondary endpoints. OS is a critical endpoint for demonstrating efficacy of a new drug; CR+CRh allows for the assessment of the total objective response rate in this population. The primary analysis of all secondary efficacy endpoints will be a re-randomization test that calculates the p-value by re-randomizing and allocating patients to treatments. For OS, the re-randomization will use a stratified Cox proportional hazard test-statistic. For CR+CRh, the CMH test-statistic will be used. The secondary endpoints will only be analyzed at the final analysis. Note that by design an interim analysis has been foreseen for OS, using an O'Brien-Fleming type Lan-DeMets boundary for efficacy. When the number of required deaths is observed, all secondary endpoints will be analyzed. The number of required deaths for a final analysis of OS is 394.

For the primary analyses, no imputations will be done and data will be analyzed as observed, with patients without response assessment set to non-responder. Multiple-imputation will be used in secondary or sensitivity analyses, whenever necessary, to impute the missing efficacy endpoint data. The SAP will provide further details on these analyses that aim at understanding the impact of missing on the estimated treatment effects.

Sub-group analyses will be carried out with a descriptive intent. Treatment effects will be estimated and tested for important baseline factors, including the factors used in the treatment allocation procedure (performance status, age and refractory versus relapsed disease), and displayed as forest plots. Interaction tests will be carried between treatment and each of these prognostic factors.

The randomized treatment arms will first be compared in terms of CR; then, conditionally on the comparison of CR reaching statistical significance, the randomized treatment arms will be compared in terms of the two key secondary endpoints, OS and CR+CRh. A Hochberg procedure will be used to adjust the significance level to allow for multiple comparisons. The Hochberg testing procedure will proceed as follows: Let p1 and p2 be the p-values of the two key secondary endpoints. The two p-values will be arranged such that p1≤p2. If p2≤0.023, then both key secondary endpoints will be declared significant. If p2 >0.023 and p1≤0.0115, then the significance of the endpoint corresponding to p1 will be claimed.

Changes over time in QLQ-C30 Global Health Status/Quality of Life (QOL) scores will be compared between treatment groups using a restricted maximum likelihood-based mixed model for repeated measures under the assumption of missing at random (Mallinckrodt, C H et al., “Recommendations for the primary analysis of continuous endpoints in longitudinal clinical trials,” Drug Inf J., 2008, 42, 303-319). As recommended, sensitivity analyses will be carried out to account for possible non-ignorable missing data (Mallinckrodt, C H et al.; Bell, M L and Fairclough, D L, “Practical and statistical issues in missing data for longitudinal patient reported outcomes,” Stat Methods Med Res, 2013, 0, 1-20). The minimal clinical important difference (MID) will help to interpret any observed differences between treatment groups. The MID for between-group differences on the QLQ-C30 Global Health Scale/QoL is 5 points according to the literature.

The determination of the positive and negative predictive value of the gene expression signature found from studying patients in the Phase I study CCCWFU 22112. Baseline bone marrow aspirate/biopsy samples will be collected in a voluntary fashion and analyzed by RNA sequencing to determine the positive and negative predictive value of the response signature determined by a similar analysis of patient samples from study CCCWFU 22112. Efficacy and safety analyses per gene mutations (these may include FLT3, IDH1/2, TP53, CEBPα, NPM1, etc) will also be assessed.

PK analysis for dose/exposure-response will be explored for clinical efficacy and safety.

Unstained slides from baseline and time of disease progression biopsies will be used for IHC staining for PDKs, PDH, KGDH, SOD2 and CD79a. Staining will be reviewed and scored by a pathologist blinded to treatment outcomes. They will also be used as a source of material for RNA and whole exome sequencing should the bone marrow aspirate/biopsy material be inadequate.

The assessment of safety will be mainly on the frequency of adverse events based on the NCI Common Terminology Criteria for Adverse Events (NCI CTCAE) version 5.0 or later. Adverse events will be coded according to the current MedDRA version. The safety outcomes will include the occurrence of at least one serious adverse event, of at least one grade 3/4 adverse event, and of at least one adverse event requiring the discontinuation of study treatment.

The sample size calculation is based on an improvement in complete remission rate (CR) from 26% in the control arm to 39% in the experimental arm (a 13% absolute increase, or a 50% relative increase), based on clinical efficacy data from Phase I study CCCWFU 22112 in AML patients. For a power of 80%, 500 patients need to be evaluated for response. This number allows for two interim analyses to be performed as detailed in the interim analysis plan.

The study is also powered to detect a clinically meaningful difference in overall survival (OS), with an expected median overall survival equal to 5.2 months in the control arm vs. 6.9 months in the experimental arm, i.e. a hazard ratio equal to 0.75 assuming exponential survival distributions. For a power of 80%, 394 events need to be observed. This number allows for one interim analysis, but is merely added to safeguard the type I error as no interim analysis for OS is planned (as detailed in the interim analysis plan). Assuming an accrual rate of 15 patients per month and a common dropout rate of 10% at 3 years, a sample size of 500 patients will provide a power of 80% for the OS analysis 36 months after the first randomization. Note that all secondary endpoints will be analyzed after 394 deaths are observed.

Finally, the study has more than 86% power to detect a clinically meaningful difference in CR+CRh rate (complete remission with or without complete hematologic recovery) at the final analysis, with an expected rate equal to 33% in the control arm vs. 47% in the experimental arm. Similarly, as for OS, this sample size allows for 1 interim analysis although there is no intent to analyze CR+CRh at interim.

Patients will be randomized in a 1:1 ratio to the experimental treatment or control, using a dynamic minimization procedure based on the methodology described by Pocock and Simon (Pocock S J and Simon R, “Sequential treatment assignment with balancing for prognostic factors in the controlled clinical trial.” Biometrics 1975, 31, 103-105). The minimization algorithm will use the range method to minimize overall imbalances between the treatment arms with respect to center and important prognostic factors (prior therapy, disease type, age, cytogenetic risk category, performance status). A stochastic minimization will be used so that no treatment allocation is deterministic (Buyse M, 2000, supra).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method for treating acute myeloid leukemia in a patient in need thereof, comprising the step of administering to the patient:
a. devimistat,
b. cytarabine, and
c. mitoxantrone;
pursuant to an induction cycle of 14 days, wherein during the induction cycle the devimistat is administered as a single daily dose of about 2,000 mg/m² on each of days 1, 2, 3, 4, and 5, the cytarabine is administered in five doses of about 1.0 g/m² each every 12 hours beginning on day 3, and the mitoxantrone is administered as a single daily dose of about 6 mg/m² on each of days 3, 4, and 5, in order to treat the acute myeloid leukemia.

2. A method for treating relapsed or refractory acute myeloid leukemia in a patient who is at least 50 years old, comprising the step of administering to a patient with relapsed or refractory acute myeloid leukemia who is at least 50 years old:
a. devimistat,
b. cytarabine, and
c. mitoxantrone;
pursuant to an induction cycle of 14 days, wherein during the induction cycle the devimistat is administered as a single daily dose of about 2,000 mg/m² on each of days 1, 2, 3, 4, and 5, the cytarabine is administered in five doses of about 1.0 g/m² each every 12 hours beginning on day 3, and the mitoxantrone is administered as a single daily dose of about 6 mg/m² on each of days 3, 4, and 5, in order to treat the relapsed or refractory acute myeloid leukemia.

3. A method for treating relapsed or refractory acute myeloid leukemia in a patient who is at least 50 years old, comprising the step of administering to a patient with relapsed or refractory acute myeloid leukemia who is at least 50 years old devimistat, cytarabine, and mitoxantrone pursuant to an induction cycle of 14 days, wherein during the induction cycle a) the devimistat is administered as a single daily dose of about 2,000 mg/m² on each of days 1, 2, 3, 4, and 5, wherein each devimistat dose being administered over 2 hours as a central line IV infusion, b) the cytarabine is administered in five doses of about 1.0 g/m² each every 12 hours beginning on day 3 after completion of the devimistat infusion, wherein each cytarabine dose is administered over 3 hours as a central line IV infusion, and c) the mitoxantrone is administered as a single daily dose of about 6 mg/m² on each of days 3, 4, and 5 after completion of the cytarabine infusion, wherein each mitoxantrone dose is administered over 15 minutes as a central line IV infusion.

4. The method of any of claims 1-3, further comprising the step of repeating the induction cycle.

5. The method of any of claims 1-3, further comprising the step of subsequently administering to the patient:
a. devimistat,
b. cytarabine, and
c. mitoxantrone;
pursuant to an abbreviated induction cycle of 14 days, wherein during the abbreviated induction cycle the devimistat is administered as a single daily dose of about 2,000 mg/m² on each of days 1, 2, and 3, the cytarabine is administered in three doses of about 1.0 g/m² each every 12 hours beginning on day 2, and the mitoxantrone is administered as a single daily dose of about 6 mg/m² on each of days 2 and 3.

6. The method of any one of claims 1-5, further comprising the step of subsequently administering to the patient:
a. devimistat,
b. cytarabine, and
c. mitoxantrone;
pursuant to consolidation cycle of 14 days, wherein during the consolidation cycle the devimistat is administered as a single daily dose of about 2,000 mg/m² on each of days 1, 2, and 3, the cytarabine is administered in three doses of about 1.0 g/m² each every 12 hours beginning on day 2, and the mitoxantrone is administered as a single daily dose of about 6 mg/m² on each of days 2 and 3.

7. The method of claim 6, further comprising the step of repeating the consolidation cycle.

8. The method of any one of claims 1-6, further comprising the step of subsequently administering to the patient devimistat pursuant to maintenance cycle of 28 days, wherein during the maintenance cycle the devimistat is administered as a single daily dose of about 2,500 mg/m² on each of days 1, 2, 3, 4, and 5.

9. The method of claim 8, wherein the maintenance cycle is repeated at least once.

10. The method of any one of claims 1-9, wherein the patient is at least 60 years old.

11. The method of any one of claims 1-10, wherein the mitoxantrone is administered intravenously to the patient as a solution prepared from its dihydrochloride salt.

12. The method of any one of claims 1-11, wherein the devimistat is administered intravenously to the patient as a solution prepared by
a. providing a 50 mg/mL solution of devimistat in 1 M aqueous triethanolamine; and
b. diluting the 50 mg/mL solution with sterile 5% dextrose for injection (D5W) to a concentration as low as about 12.5 mg/mL.

13. The method of claim 12, wherein the 50 mg/mL solution is diluted with D5W to a concentration of about 12.5 mg/mL.

14. The method of any one of claims 1-13, wherein the first, third, and fifth cytarabine doses are started less than or equal to 30 minutes after completion of the day 3, day 4, and day 5 devimistat infusions, and the mitoxantrone doses are started less than or equal to 30 minutes after completing the first, third, and fifth cytarabine infusions.

15. A medical kit, comprising (i) devimistat and (ii) instructions for treating acute myeloid leukemia in a patient according to the method of any preceding claim.
